SEQUENCE LISTING (35 sequences)

SEQ ID NO: 1 - protein, 253 aa, Artificial Sequence
  SPamyL-PmaCA-CA3
MKQQKRLYARLLTLLFALIFLLPHSAAAAGGGWSYHGEHGPEHWGDLKDEYIMCKIGKNQSPVDINRIVDAKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYSGSLTTPPCSEGVRWIVMEEEMEMSKEQIEKFRKIMGGDTNRPVQPLNARMIMEK

SEQ ID NO: 2 - DNA, 762 bp, Artificial Sequence
  SPamyL-PmaCA-CA3
atgaaacaacagaaaagactgtatgcacgcctgcttacattactgtttgctcttatttttcttttaccgcattcagcagcggctgccggcggaggatggagctatcatggcgaacatggacctgaacattggggtgacctgaaagacgaatatattatgtgcaaaatcggcaaaaatcaatcaccggttgatattaacagaatcgtggatgcaaaacttaaaccgatcaaaatcgaatatcgcgcaggagcgacaaaagtcctgaacaacggccatacaatcaaagtttcttatgaaccgggatcatatattgttgtggatggcatcaaatttgaattaaaacaatttcattttcatgcaccgagcgaacataaactgaaaggacagcattatccgtttgaagctcattttgttcatgccgataaacatggcaatctggctgtcatcggagttttctttaaagaaggcagagaaaacccgattcttgaaaaaatctggaaagtgatgccggaaaatgccggcgaagaagtcaaattagcacataaaatcaacgcggaagatttactgccgaaagatagagattattatcgctattcaggaagcctgacaacaccgccgtgcagcgaaggcgtgagatggatcgtcatggaagaagaaatggaaatgtctaaagaacagatcgaaaaatttcgcaaaatcatgggaggcgatacgaaccgtcctgtgcagccgttgaatgcgagaatgattatggaaaaataa

SEQ ID NO: 3 - protein, 253 aa, Artificial Sequence
  SPamyL-PmaCA-CA3mut1
MKQQKRLYARLLTLLFALIFLLPHSAAAAGGGWSYHGEHGPEHWGDLKDEYIMCKIGKNQSPVDINRIVDAKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYSGSLTTPPCSECVRWIVMEEEMEMSKEQIEKFRKIMGGDTNRPVQPLCARMIMEK

SEQ ID NO: 4 - DNA, 762 bp, Artificial Sequence
  SPamyL-PmaCA-CA3mut1
atgaaacaacagaaaagactgtatgcacgcctgcttacattactgtttgctcttatttttcttttaccgcattcagcagcggctgccggcggaggatggagctatcatggcgaacatggacctgaacattggggtgacctgaaagacgaatatattatgtgcaaaatcggcaaaaatcaatcaccggttgatattaacagaatcgtggatgcaaaacttaaaccgatcaaaatcgaatatcgcgcaggagcgacaaaagtcctgaacaacggccatacaatcaaagtttcttatgaaccgggatcatatattgttgtggatggcatcaaatttgaattaaaacaatttcattttcatgcaccgagcgaacataaactgaaaggacagcattatccgtttgaagctcattttgttcatgccgataaacatggcaatctggctgtcatcggagttttctttaaagaaggcagagaaaacccgattcttgaaaaaatctggaaagtgatgccggaaaatgccggcgaagaagtcaaattagcacataaaatcaacgcggaagatttactgccgaaagatagagattattatcgctattcaggaagcctgacaacaccgccgtgcagcgaatgcgtgagatggatcgtcatggaagaagaaatggaaatgtctaaagaacagatcgaaaaatttcgcaaaatcatgggaggcgatacgaaccgtcctgtgcagccgttgtgtgcgagaatgattatggaaaaataa

SEQ ID NO: 5 - protein, 253 aa, Artificial Sequence
  SPamyL-PmaCA-CA3mut2
MKQQKRLYARLLTLLFALIFLLPHSAAAAGGGWSYHGEHGPEHWGDLKDEYIMCKIGKNQSPVDINRIVDCKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYSGSLTTPPCSEGVRWIVMEEEMEMSKEQIEKFRKIMGGDTNRPVQPLNARMIMEK

SEQ ID NO: 6 - DNA, 762 bp, Artificial Sequence
  SPamyL-PmaCA-CA3mut2
atgaaacaacagaaaagactgtatgcacgcctgcttacattactgtttgctcttatttttcttttaccgcattcagcagcggctgccggcggaggatggagctatcatggcgaacatggacctgaacattggggtgacctgaaagacgaatatattatgtgcaaaatcggcaaaaatcaatcaccggttgatattaacagaatcgtggattgtaaacttaaaccgatcaaaatcgaatatcgcgcaggagcgacaaaagtcctgaacaacggccatacaatcaaagtttcttatgaaccgggatcatatattgttgtggatggcatcaaatttgaattaaaacaatttcattttcatgcaccgagcgaacataaactgaaaggacagcattatccgtttgaagctcattttgttcatgccgataaacatggcaatctggctgtcatcggagttttctttaaagaaggcagagaaaacccgattcttgaaaaaatctggaaagtgatgccggaaaatgccggcgaagaagtcaaattagcacataaaatcaacgcggaagatttactgccgaaagatagagattattatcgctattcaggaagcctgacaacaccgccgtgcagcgaaggcgtgagatggatcgtcatggaagaagaaatggaaatgtctaaagaacagatcgaaaaatttcgcaaaatcatgggaggcgatacgaaccgtcctgtgcagccgttgaatgcgagaatgattatggaaaaataa

SEQ ID NO: 7 - protein, 253 aa, Artificial Sequence
  SPamyL-PmaCA-CA3mut3
MKQQKRLYARLLTLLFALIFLLPHSAAAAGGGWSYHGEHGPEHWGDLKDEYIMCKIGKNQSPVDINRIVDAKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYCGSLTTPPCSEGVRWIVMEEEMEMSKEQIEKFRKIMGGDTNRPVQPLCARMIMEK

SEQ ID NO: 8 - DNA, 762 bp, Artificial Sequence
  SPamyL-PmaCA-CA3mut3
atgaaacaacagaaaagactgtatgcacgcctgcttacattactgtttgctcttatttttcttttaccgcattcagcagcggctgccggcggaggatggagctatcatggcgaacatggacctgaacattggggtgacctgaaagacgaatatattatgtgcaaaatcggcaaaaatcaatcaccggttgatattaacagaatcgtggatgcaaaacttaaaccgatcaaaatcgaatatcgcgcaggagcgacaaaagtcctgaacaacggccatacaatcaaagtttcttatgaaccgggatcatatattgttgtggatggcatcaaatttgaattaaaacaatttcattttcatgcaccgagcgaacataaactgaaaggacagcattatccgtttgaagctcattttgttcatgccgataaacatggcaatctggctgtcatcggagttttctttaaagaaggcagagaaaacccgattcttgaaaaaatctggaaagtgatgccggaaaatgccggcgaagaagtcaaattagcacataaaatcaacgcggaagatttactgccgaaagatagagattattatcgctattgtggaagcctgacaacaccgccgtgcagcgaaggcgtgagatggatcgtcatggaagaagaaatggaaatgtctaaagaacagatcgaaaaatttcgcaaaatcatgggaggcgatacgaaccgtcctgtgcagccgttgtgtgcgagaatgattatggaaaaataa

SEQ ID NO: 9 - protein, 253 aa, Artificial Sequence
  SPamyL-PmaCA-CA3mut23
MKQQKRLYARLLTLLFALIFLLPHSAAAAGGGWSYHGEHGPEHWGDLKDEYIMCKIGKNQSPVDINRIVDCKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYCGSLTTPPCSEGVRWIVMEEEMEMSKEQIEKFRKIMGGDTNRPVQPLNCRMIMEK

SEQ ID NO: 10 - DNA, 762 bp, Artificial Sequence
  SPamyL-PmaCA-CA3mut23
atgaaacaacagaaaagactgtatgcacgcctgcttacattactgtttgctcttatttttcttttaccgcattcagcagcggctgccggcggaggatggagctatcatggcgaacatggacctgaacattggggtgacctgaaagacgaatatattatgtgcaaaatcggcaaaaatcaatcaccggttgatattaacagaatcgtggattgtaaacttaaaccgatcaaaatcgaatatcgcgcaggagcgacaaaagtcctgaacaacggccatacaatcaaagtttcttatgaaccgggatcatatattgttgtggatggcatcaaatttgaattaaaacaatttcattttcatgcaccgagcgaacataaactgaaaggacagcattatccgtttgaagctcattttgttcatgccgataaacatggcaatctggctgtcatcggagttttctttaaagaaggcagagaaaacccgattcttgaaaaaatctggaaagtgatgccggaaaatgccggcgaagaagtcaaattagcacataaaatcaacgcggaagatttactgccgaaagatagagattattatcgctattgtggaagcctgacaacaccgccgtgcagcgaaggcgtgagatggatcgtcatggaagaagaaatggaaatgtctaaagaacagatcgaaaaatttcgcaaaatcatgggaggcgatacgaaccgtcctgtgcagccgttgtgtgcgagaatgattatggaaaaataa

SEQ ID NO: 11 - protein, 255 aa, Artificial Sequence
  SPamyL-LOGACA-CA4
MKQQKRLYARLLTLLFALIFLLPHSAAAAGGVGHWSYHGETGPQHWGDLKNEYIMCKIGKNQSPVDISRIVEAELEKIKINYSSGGSSITNNGHTIKVSYEPGSYIIVDGIRFELKQFHFHAPSEHTIKGKSYPFEAHFVHADKDGNLAVIGVIFKEGKKNPIIEKIWENLPEAGKTIKLAHKINAYDLLPKKKKYYRYSGSLTTPPCSEGVRWIVMEEEMELSKEQIEKFRKLMGGDTNRPVQPLNARMIMEMD

SEQ ID NO: 12 - DNA, 768 bp, Artificial Sequence
  SPamyL-LOGACA-CA4
atgaaacaacagaaaagactgtatgcacgcctgcttacattactgtttgctcttatttttcttttaccgcattcagcagcggctgccggcggagttggacattggtcttatcatggcgaaacaggaccgcaacattggggcgatctgaaaaacgaatacatcatgtgcaaaatcggcaaaaaccagtcaccggtggatattagcagaatcgtcgaagctgaacttgaaaaaatcaaaatcaactattcaagcggcggatcttcaatcacaaacaacggacatacaatcaaagtttcttatgaaccgggatcatatattatcgtggatggcattcgctttgaattaaaacaatttcattttcatgccccgagcgaacatacaatcaaaggcaaatcttatccgtttgaagcacattttgtccatgcggataaagatggcaatctggcagttattggagtgatctttaaagaaggcaagaaaaatccgatcatcgaaaaaatttgggaaaacttaccggaagcgggcaaaacaatcaaactggctcataaaatcaacgcctatgatctgcttccgaaaaagaaaaaatactacagatacagcggatctcttacaacaccgccgtgttcagaaggcgtccgctggattgttatggaagaagaaatggaacttagcaaagaacaaatcgaaaaatttagaaaactgatgggcggagatacaaatagaccggttcagccgttaaacgctcgcatgattatggaaatggattaa

SEQ ID NO: 13 - protein, 255 aa, Artificial Sequence
  SPamyL-LOGACA-CA4mut2
MKQQKRLYARLLTLLFALIFLLPHSAAAAGGVGHWSYHGETGPQHWGDLKNEYIMCKIGKNQSPVDISRIVECELEKIKINYSSGGSSITNNGHTIKVSYEPGSYIIVDGIRFELKQFHFHAPSEHTIKGKSYPFEAHFVHADKDGNLAVIGVIFKEGKKNPIIEKIWENLPEAGKTIKLAHKINAYDLLPKKKKYYRYSGSLTTPPCSEGVRWIVMEEEMELSKEQIEKFRKLMGGDTNRPVQPLNARMIMEMD

SEQ ID NO: 14 - DNA, 768 bp, Artificial Sequence
  SPamyL-LOGACA-CA4mut2
atgaaacaacagaaaagactgtatgcacgcctgcttacattactgtttgctcttatttttcttttaccgcattcagcagcggctgccggcggagttggacattggtcttatcatggcgaaacaggaccgcaacattggggcgatctgaaaaacgaatacatcatgtgcaaaatcggcaaaaaccagtcaccggtggatattagcagaatcgtcgaatgtgaacttgaaaaaatcaaaatcaactattcaagcggcggatcttcaatcacaaacaacggacatacaatcaaagtttcttatgaaccgggatcatatattatcgtggatggcattcgctttgaattaaaacaatttcattttcatgccccgagcgaacatacaatcaaaggcaaatcttatccgtttgaagcacattttgtccatgcggataaagatggcaatctggcagttattggagtgatctttaaagaaggcaagaaaaatccgatcatcgaaaaaatttgggaaaacttaccggaagcgggcaaaacaatcaaactggctcataaaatcaacgcctatgatctgcttccgaaaaagaaaaaatactacagatacagcggatctcttacaacaccgccgtgttcagaaggcgtccgctggattgttatggaagaagaaatggaacttagcaaagaacaaatcgaaaaatttagaaaactgatgggcggagatacaaatagaccggttcagccgttaaacgctcgcatgattatggaaatggattaa

SEQ ID NO: 15 - protein, 255 aa, Artificial Sequence
  SPamyL-LOGACA-CA4mut3
MKQQKRLYARLLTLLFALIFLLPHSAAAAGGVGHWSYHGETGPQHWGDLKNEYIMCKIGKNQSPVDISRIVEAELEKIKINYSSGGSSITNNGHTIKVSYEPGSYIIVDGIRFELKQFHFHAPSEHTIKGKSYPFEAHFVHADKDGNLAVIGVIFKEGKKNPIIEKIWENLPEAGKTIKLAHKINAYDLLPKKKKYYRYCGSLTTPPCSEGVRWIVMEEEMELSKEQIEKFRKLMGGDTNRPVQPLNCRMIMEMD

SEQ ID NO: 16 - DNA, 768 bp, Artificial Sequence
  SPamyL-LOGACA-CA4mut3
atgaaacaacagaaaagactgtatgcacgcctgcttacattactgtttgctcttatttttcttttaccgcattcagcagcggctgccggcggagttggacattggtcttatcatggcgaaacaggaccgcaacattggggcgatctgaaaaacgaatacatcatgtgcaaaatcggcaaaaaccagtcaccggtggatattagcagaatcgtcgaagctgaacttgaaaaaatcaaaatcaactattcaagcggcggatcttcaatcacaaacaacggacatacaatcaaagtttcttatgaaccgggatcatatattatcgtggatggcattcgctttgaattaaaacaatttcattttcatgccccgagcgaacatacaatcaaaggcaaatcttatccgtttgaagcacattttgtccatgcggataaagatggcaatctggcagttattggagtgatctttaaagaaggcaagaaaaatccgatcatcgaaaaaatttgggaaaacttaccggaagcgggcaaaacaatcaaactggctcataaaatcaacgcctatgatctgcttccgaaaaagaaaaaatactacagatactgcggatctcttacaacaccgccgtgttcagaaggcgtccgctggattgttatggaagaagaaatggaacttagcaaagaacaaatcgaaaaatttagaaaactgatgggcggagatacaaatagaccggttcagccgttaaactgtcgcatgattatggaaatggattaa

SEQ ID NO: 17 - protein, 255 aa, Artificial Sequence
  SPamyL-LOGACA-CA4mut23
MKQQKRLYARLLTLLFALIFLLPHSAAAAGGVGHWSYHGETGPQHWGDLKNEYIMCKIGKNQSPVDISRIVECELEKIKINYSSGGSSITNNGHTIKVSYEPGSYIIVDGIRFELKQFHFHAPSEHTIKGKSYPFEAHFVHADKDGNLAVIGVIFKEGKKNPIIEKIWENLPEAGKTIKLAHKINAYDLLPKKKKYYRYCGSLTTPPCSEGVRWIVMEEEMELSKEQIEKFRKLMGGDTNRPVQPLNCRMIMEMD

SEQ ID NO: 18 - DNA, 768 bp, Artificial Sequence
  SPamyL-LOGACA-CA4mut23
atgaaacaacagaaaagactgtatgcacgcctgcttacattactgtttgctcttatttttcttttaccgcattcagcagcggctgccggcggagttggacattggtcttatcatggcgaaacaggaccgcaacattggggcgatctgaaaaacgaatacatcatgtgcaaaatcggcaaaaaccagtcaccggtggatattagcagaatcgtcgaatgtgaacttgaaaaaatcaaaatcaactattcaagcggcggatcttcaatcacaaacaacggacatacaatcaaagtttcttatgaaccgggatcatatattatcgtggatggcattcgctttgaattaaaacaatttcattttcatgccccgagcgaacatacaatcaaaggcaaatcttatccgtttgaagcacattttgtccatgcggataaagatggcaatctggcagttattggagtgatctttaaagaaggcaagaaaaatccgatcatcgaaaaaatttgggaaaacttaccggaagcgggcaaaacaatcaaactggctcataaaatcaacgcctatgatctgcttccgaaaaagaaaaaatactacagatactgcggatctcttacaacaccgccgtgttcagaaggcgtccgctggattgttatggaagaagaaatggaacttagcaaagaacaaatcgaaaaatttagaaaactgatgggcggagatacaaatagaccggttcagccgttaaactgtcgcatgattatggaaatggattaa

SEQ ID NO: 19 - protein, 255 aa, Artificial Sequence
  SPamyL-SspCA-CA5
MKQQKRLYARLLTLLFALIFLLPHSAAAAEHEWSYEGEKGPEHWAQLKPEFFWCKLKNQSPINIDKKYKVKANLPKLNLYYKTAKESEVVNNGHTIQINIKEDNTLNYLGEKYQLKQFHFHTPSEHTIEKKSYPLEIHFVHKTEDGKILVVGVMAKLGKTNKELDKILNVAPAEEGEKILDKNLNLNNLIPKDKRYMTYSGSLTTPPCTEGVRWIVLKKPISISKQQLEKLKSVMVNPNNRPVQEINSRWIIEGF

SEQ ID NO: 20 - DNA, 768 bp, Artificial Sequence
  SPamyL-SspCA-CA5
atgaaacaacagaaaagactgtatgcacgcctgcttacattactgtttgctcttatttttcttttaccgcattcagcagcggctgccgaacatgaatggtcttatgaaggcgaaaaaggaccggaacattgggcacaactgaaaccggaatttttctggtgcaaacttaaaaaccagtcaccgatcaacatcgataaaaaatacaaagttaaagctaacctgccgaaactgaacctttactacaaaacagccaaagaatcagaagttgtgaataacggacatacaatccaaatcaacatcaaagaagataacacacttaactacctgggcgaaaaataccaactgaaacagtttcattttcatacaccgagcgaacatacaatcgagaaaaaatcatacccgcttgaaatccattttgtccataaaacagaagatggcaaaatccttgtcgttggagttatggctaaactgggcaaaacaaacaaagaattagataaaattctgaacgtggcaccggcggaagaaggagaaaaaatcttagataaaaacctgaacctgaacaacctgatcccgaaagataaaagatacatgacatactcaggaagccttacaacaccgccgtgtacagaaggcgttcgctggatcgtgctgaaaaaaccgatctctatttcaaaacaacagctggaaaaacttaaatcagtgatggtcaatccgaataacagaccggtccaggaaattaacagccgctggattatcgaaggcttttaa

SEQ ID NO: 21 - protein, 255 aa, Artificial Sequence
  SPamyL-SspCA-CA5mut2
MKQQKRLYARLLTLLFALIFLLPHSAAAAEHEWSYEGEKGPEHWAQLKPEFFWCKLKNQSPINIDKKYKVKCNLPKLNLYYKTAKESEVVNNGHTIQINIKEDNTLNYLGEKYQLKQFHFHTPSEHTIEKKSYPLEIHFVHKTEDGKILVVGVMAKLGKTNKELDKILNVAPAEEGEKILDKNLNLNNLIPKDKRYMTYSGSLTTPPCTEGVRWIVLKKPISISKQQLEKLKSVMVNPNNRPVQEINSRWIIEGF

SEQ ID NO: 22 - DNA, 768 bp, Artificial Sequence
  SPamyL-SspCA-CA5mut2
atgaaacaacagaaaagactgtatgcacgcctgcttacattactgtttgctcttatttttcttttaccgcattcagcagcggctgccgaacatgaatggtcttatgaaggcgaaaaaggaccggaacattgggcacaactgaaaccggaatttttctggtgcaaacttaaaaaccagtcaccgatcaacatcgataaaaaatacaaagttaaatgtaacctgccgaaactgaacctttactacaaaacagccaaagaatcagaagttgtgaataacggacatacaatccaaatcaacatcaaagaagataacacacttaactacctgggcgaaaaataccaactgaaacagtttcattttcatacaccgagcgaacatacaatcgagaaaaaatcatacccgcttgaaatccattttgtccataaaacagaagatggcaaaatccttgtcgttggagttatggctaaactgggcaaaacaaacaaagaattagataaaattctgaacgtggcaccggcggaagaaggagaaaaaatcttagataaaaacctgaacctgaacaacctgatcccgaaagataaaagatacatgacatactcaggaagccttacaacaccgccgtgtacagaaggcgttcgctggatcgtgctgaaaaaaccgatctctatttcaaaacaacagctggaaaaacttaaatcagtgatggtcaatccgaataacagaccggtccaggaaattaacagccgctggattatcgaaggcttttaa

SEQ ID NO: 23 - protein, 255 aa, Artificial Sequence
  SPamyL-SspCA-CA5mut3
MKQQKRLYARLLTLLFALIFLLPHSAAAAEHEWSYEGEKGPEHWAQLKPEFFWCKLKNQSPINIDKKYKVKANLPKLNLYYKTAKESEVVNNGHTIQINIKEDNTLNYLGEKYQLKQFHFHTPSEHTIEKKSYPLEIHFVHKTEDGKILVVGVMAKLGKTNKELDKILNVAPAEEGEKILDKNLNLNNLIPKDKRYMTYCGSLTTPPCTEGVRWIVLKKPISISKQQLEKLKSVMVNPNNRPVQEINCRWIIEGF

SEQ ID NO: 24 - DNA, 768 bp, Artificial Sequence
  SPamyL-SspCA-CA5mut3
atgaaacaacagaaaagactgtatgcacgcctgcttacattactgtttgctcttatttttcttttaccgcattcagcagcggctgccgaacatgaatggtcttatgaaggcgaaaaaggaccggaacattgggcacaactgaaaccggaatttttctggtgcaaacttaaaaaccagtcaccgatcaacatcgataaaaaatacaaagttaaagctaacctgccgaaactgaacctttactacaaaacagccaaagaatcagaagttgtgaataacggacatacaatccaaatcaacatcaaagaagataacacacttaactacctgggcgaaaaataccaactgaaacagtttcattttcatacaccgagcgaacatacaatcgagaaaaaatcatacccgcttgaaatccattttgtccataaaacagaagatggcaaaatccttgtcgttggagttatggctaaactgggcaaaacaaacaaagaattagataaaattctgaacgtggcaccggcggaagaaggagaaaaaatcttagataaaaacctgaacctgaacaacctgatcccgaaagataaaagatacatgacatactgcggaagccttacaacaccgccgtgtacagaaggcgttcgctggatcgtgctgaaaaaaccgatctctatttcaaaacaacagctggaaaaacttaaatcagtgatggtcaatccgaataacagaccggtccaggaaattaactgtcgctggattatcgaaggcttttaa

SEQ ID NO: 25 - protein, 255 aa, Artificial Sequence
  SPamyL-SspCA-CA5mut23
MKQQKRLYARLLTLLFALIFLLPHSAAAAEHEWSYEGEKGPEHWAQLKPEFFWCKLKNQSPINIDKKYKVKCNLPKLNLYYKTAKESEVVNNGHTIQINIKEDNTLNYLGEKYQLKQFHFHTPSEHTIEKKSYPLEIHFVHKTEDGKILVVGVMAKLGKTNKELDKILNVAPAEEGEKILDKNLNLNNLIPKDKRYMTYCGSLTTPPCTEGVRWIVLKKPISISKQQLEKLKSVMVNPNNRPVQEINCRWIIEGF

SEQ ID NO: 26 - DNA, 768 bp, Artificial Sequence
  SPamyL-SspCA-CA5mut23
atgaaacaacagaaaagactgtatgcacgcctgcttacattactgtttgctcttatttttcttttaccgcattcagcagcggctgccgaacatgaatggtcttatgaaggcgaaaaaggaccggaacattgggcacaactgaaaccggaatttttctggtgcaaacttaaaaaccagtcaccgatcaacatcgataaaaaatacaaagttaaatgtaacctgccgaaactgaacctttactacaaaacagccaaagaatcagaagttgtgaataacggacatacaatccaaatcaacatcaaagaagataacacacttaactacctgggcgaaaaataccaactgaaacagtttcattttcatacaccgagcgaacatacaatcgagaaaaaatcatacccgcttgaaatccattttgtccataaaacagaagatggcaaaatccttgtcgttggagttatggctaaactgggcaaaacaaacaaagaattagataaaattctgaacgtggcaccggcggaagaaggagaaaaaatcttagataaaaacctgaacctgaacaacctgatcccgaaagataaaagatacatgacatactgcggaagccttacaacaccgccgtgtacagaaggcgttcgctggatcgtgctgaaaaaaccgatctctatttcaaaacaacagctggaaaaacttaaatcagtgatggtcaatccgaataacagaccggtccaggaaattaactgtcgctggattatcgaaggcttttaa

SEQ ID NO: 27 - protein, 266 aa, Artificial Sequence
  SPamyL-SazCA-CA6FL
MKQQKRLYARLLTLLFALIFLLPHSAAAAGEHAILQKNAEVHHWSYEGENGPENWAKLNPEYFWCNLKNQSPVDISDNYKVHAKLEKLHINYNKAVNPEIVNNGHTIQVNVLEDFKLNIKGKEYHLKQFHFHAPSEHTVNGKYYPLEMHLVHKDKDGNIAVIGVFFKEGKANPELDKVFKNALKEEGSKVFDGSININALLPPVKNYYTYSGSLTTPPCTEGVLWIVLKQPITASKQQIELFKSIMKHNNNRPTQPINSRYILESN

SEQ ID NO: 28 - DNA, 801 bp, Artificial Sequence
  SPamyL-SazCA-CA6FL
atgaaacaacagaaaagactgtatgcacgcctgcttacattactgtttgctcttatttttcttttaccgcattcagcagcggctgccggcgaacatgcaattctgcagaaaaatgcggaagtccatcattggagctatgaaggcgaaaacggaccggaaaattgggccaaactgaacccggaatacttttggtgcaaccttaaaaaccagtctccggtcgatatttcagataactacaaagttcatgccaaactggaaaaactgcatatcaactacaacaaagcagttaacccggaaattgtgaataacggacatacaatccaagttaacgtgttagaagattttaaactgaacatcaaaggcaaagaataccatcttaaacagtttcattttcatgctccgtctgaacatacagtgaacggcaaatattatccgcttgaaatgcatctggtccataaagataaagatggcaacattgcagtcatcggagttttctttaaagaaggcaaagcgaacccggaacttgataaagtttttaaaaacgctctgaaagaagaaggaagcaaagtgtttgatggctctattaacatcaatgcgctgcttccgccggttaaaaactactacacatactcaggaagcttaacaacaccgccgtgtacagaaggcgtgctgtggattgtccttaaacaaccgatcacagcttctaaacaacagattgaactgtttaaatcaatcatgaaacataacaacaatagaccgacacagccgattaactcacgctatatcctggaaagcaattaa

SEQ ID NO: 29 - protein, 266 aa, Artificial Sequence
  SPamyL-SazCA-CA6FLmut2
MKQQKRLYARLLTLLFALIFLLPHSAAAAGEHAILQKNAEVHHWSYEGENGPENWAKLNPEYFWCNLKNQSPVDISDNYKVHCKLEKLHINYNKAVNPEIVNNGHTIQVNVLEDFKLNIKGKEYHLKQFHFHAPSEHTVNGKYYPLEMHLVHKDKDGNIAVIGVFFKEGKANPELDKVFKNALKEEGSKVFDGSININALLPPVKNYYTYSGSLTTPPCTEGVLWIVLKQPITASKQQIELFKSIMKHNNNRPTQPINSRYILESN

SEQ ID NO: 30 - DNA, 801 bp, Artificial Sequence
  SPamyL-SazCA-CA6FLmut2
atgaaacaacagaaaagactgtatgcacgcctgcttacattactgtttgctcttatttttcttttaccgcattcagcagcggctgccggcgaacatgcaattctgcagaaaaatgcggaagtccatcattggagctatgaaggcgaaaacggaccggaaaattgggccaaactgaacccggaatacttttggtgcaaccttaaaaaccagtctccggtcgatatttcagataactacaaagttcattgtaaactggaaaaactgcatatcaactacaacaaagcagttaacccggaaattgtgaataacggacatacaatccaagttaacgtgttagaagattttaaactgaacatcaaaggcaaagaataccatcttaaacagtttcattttcatgctccgtctgaacatacagtgaacggcaaatattatccgcttgaaatgcatctggtccataaagataaagatggcaacattgcagtcatcggagttttctttaaagaaggcaaagcgaacccggaacttgataaagtttttaaaaacgctctgaaagaagaaggaagcaaagtgtttgatggctctattaacatcaatgcgctgcttccgccggttaaaaactactacacatactcaggaagcttaacaacaccgccgtgtacagaaggcgtgctgtggattgtccttaaacaaccgatcacagcttctaaacaacagattgaactgtttaaatcaatcatgaaacataacaacaatagaccgacacagccgattaactcacgctatatcctggaaagcaattaa

SEQ ID NO: 31 - protein, 266 aa, Artificial Sequence
  SPamyL-SazCA-CA6FLmut3
MKQQKRLYARLLTLLFALIFLLPHSAAAAGEHAILQKNAEVHHWSYEGENGPENWAKLNPEYFWCNLKNQSPVDISDNYKVHAKLEKLHINYNKAVNPEIVNNGHTIQVNVLEDFKLNIKGKEYHLKQFHFHAPSEHTVNGKYYPLEMHLVHKDKDGNIAVIGVFFKEGKANPELDKVFKNALKEEGSKVFDGSININALLPPVKNYYTYCGSLTTPPCTEGVLWIVLKQPITASKQQIELFKSIMKHNNNRPTQPINCRYILESN

SEQ ID NO: 32 - DNA, 801 bp, Artificial Sequence
  SPamyL-SazCA-CA6FLmut3
atgaaacaacagaaaagactgtatgcacgcctgcttacattactgtttgctcttatttttcttttaccgcattcagcagcggctgccggcgaacatgcaattctgcagaaaaatgcggaagtccatcattggagctatgaaggcgaaaacggaccggaaaattgggccaaactgaacccggaatacttttggtgcaaccttaaaaaccagtctccggtcgatatttcagataactacaaagttcatgccaaactggaaaaactgcatatcaactacaacaaagcagttaacccggaaattgtgaataacggacatacaatccaagttaacgtgttagaagattttaaactgaacatcaaaggcaaagaataccatcttaaacagtttcattttcatgctccgtctgaacatacagtgaacggcaaatattatccgcttgaaatgcatctggtccataaagataaagatggcaacattgcagtcatcggagttttctttaaagaaggcaaagcgaacccggaacttgataaagtttttaaaaacgctctgaaagaagaaggaagcaaagtgtttgatggctctattaacatcaatgcgctgcttccgccggttaaaaactactacacatactgcggaagcttaacaacaccgccgtgtacagaaggcgtgctgtggattgtccttaaacaaccgatcacagcttctaaacaacagattgaactgtttaaatcaatcatgaaacataacaacaatagaccgacacagccgattaactgtcgctatatcctggaaagcaattaa

SEQ ID NO: 33 - protein, 266 aa, Artificial Sequence
  SPamyL-SazCA-CA6FLmut23
MKQQKRLYARLLTLLFALIFLLPHSAAAAGEHAILQKNAEVHHWSYEGENGPENWAKLNPEYFWCNLKNQSPVDISDNYKVHCKLEKLHINYNKAVNPEIVNNGHTIQVNVLEDFKLNIKGKEYHLKQFHFHAPSEHTVNGKYYPLEMHLVHKDKDGNIAVIGVFFKEGKANPELDKVFKNALKEEGSKVFDGSININALLPPVKNYYTYCGSLTTPPCTEGVLWIVLKQPITASKQQIELFKSIMKHNNNRPTQPINCRYILESN

SEQ ID NO: 34 - DNA, 801 bp, Artificial Sequence
  SPamyL-SazCA-CA6FLmut23
atgaaacaacagaaaagactgtatgcacgcctgcttacattactgtttgctcttatttttcttttaccgcattcagcagcggctgccggcgaacatgcaattctgcagaaaaatgcggaagtccatcattggagctatgaaggcgaaaacggaccggaaaattgggccaaactgaacccggaatacttttggtgcaaccttaaaaaccagtctccggtcgatatttcagataactacaaagttcattgtaaactggaaaaactgcatatcaactacaacaaagcagttaacccggaaattgtgaataacggacatacaatccaagttaacgtgttagaagattttaaactgaacatcaaaggcaaagaataccatcttaaacagtttcattttcatgctccgtctgaacatacagtgaacggcaaatattatccgcttgaaatgcatctggtccataaagataaagatggcaacattgcagtcatcggagttttctttaaagaaggcaaagcgaacccggaacttgataaagtttttaaaaacgctctgaaagaagaaggaagcaaagtgtttgatggctctattaacatcaatgcgctgcttccgccggttaaaaactactacacatactgcggaagcttaacaacaccgccgtgtacagaaggcgtgctgtggattgtccttaaacaaccgatcacagcttctaaacaacagattgaactgtttaaatcaatcatgaaacataacaacaatagaccgacacagccgattaactgtcgctatatcctggaaagcaattaa

SEQ ID NO: 35 - protein, 29 aa, Bacillus licheniformis
MKQQKRLYARLLTLLFALIFLLPHSAAAA

(19) United States
(12) Patent Application Publication
(10) Pub. No.: US 2024/0132869 A1
(43) Pub. Date: Apr. 25, 2024

(54) ENGINEERED THERMOSTABLE CARBONIC ANHYDRASE ENZYMES

(71) Applicant: Alliance for Sustainable Energy, LLC, Golden, CO (US)

(72) Inventors: Min ZHANG, Lakewood, CO (US); Vladimir Vladimirovich LUNIN, Evergreen, CO (US); Hui WEI, Wheat Ridge, CO (US)

(21) Appl. No.: 18/546,920

(22) PCT Filed: Feb. 22, 2022
(86) PCT No.: PCT/US22/17205

§ 371 Date: Aug. 16, 2023

Related U.S. Application Data
(60) Provisional application No. 63/174,337, filed on Apr. 13, 2021.
(60) Provisional application No. 63/151,506, filed on Feb. 19, 2021.

(51) Int. Cl. C12N 9/88; B01D 53/84

(52) U.S. Cl. CPC C12N 9/88; B01D 53/84; C12Y 402/01001; B01D 2255/804; B01D 2257/504

(57) ABSTRACT

Disclosed herein are compositions and methods for making and using improved carbonic anhydrases for novel, sustainable, and low energy CO₂ waste gas scrubbing technologies that are also transformational carbon capture technologies. Embodiments of methods, systems and compositions disclosed herein include, but are not limited to, non-aqueous solvents, advanced membranes, sorbents, and cryogenic systems that significantly reduce the cost of CO₂ capture from coal and natural gas-fired power plants and industrial facilities. Methods disclosed herein reduce the energy and cost required for CO₂ separation and can be applied for both pre-combustion and post-combustion CO₂ capture.

### CROSS-REFERENCE TO RELATED APPLICATIONS

This application is a national phase entry under 35 U.S.C. § 371 and claims priority to PCT application number PCT/US/2022/017205 filed on 22 Feb. 2022. PCT/US/2022/017205 claims priority under 35 U.S.C. § 119 to U.S. provisional patent application No. 63/151,506 filed on 19 Feb. 2021 and 63/174,337 filed on 13 Apr. 2021, the contents of which are hereby incorporated in their entirety.

### CONTRACTUAL ORIGIN

The United States Government has rights in this invention under Contract No. DE-AC36-08G028308 between the United States Department of Energy and the Alliance for Sustainable Energy, LLC, the Manager and Operator of the National Renewable Energy Laboratory.

### SEQUENCE LISTING

This application contains a Sequence Listing entitled NREL_20-137_ST25.txt, filed herewith, that is 61,440 bytes in size and was created on 2 Nov. 2023.

### BACKGROUND

Energy demand continues to rise along with CO₂ emissions. Carbon Capture and Storage (CCS) plays a significant role in reducing CO₂ emissions produced from the use of fossil fuels in electricity generation and industrial processes. Bioenergy with Carbon Capture and Storage (BECCS) combines the use of biopower with greenhouse gas mitigating technology to produce energy with net-negative emissions. However, today's capture technologies are not cost-effective. Most current CCS processes rely on carbon scrubbing of flue gases with solvents like monoethanolamine (MEA) which requires energy intensive heating and cooling of the MEA to capture and release the CO₂ generated in combustion. In addition, the solvent is corrosive and suffers degradation by other species present in gas mixtures. There is a need for alternative novel scrubbing techniques that incorporate biological solutions for capturing CO₂ to improve the cost of carbon capture.

Carbonic anhydrases (CAs) are an example of convergent evolution where at least five distinct families of enzymes catalyze the same reaction but do not share significant sequence similarity or fold. Most but not all families of CA have been characterized structurally.

The chemical and enzymatic properties of CAs, like specific activity, thermal stability, and chemical stability vary greatly and have been previously targeted for improvement in industrial applications.

### SUMMARY

In an aspect, disclosed herein is a non-naturally occurring carbonic anhydrase comprising at least one mutation that results in the substitution of at least one cysteine for at least one amino acid in a naturally occurring carbonic anhydrase; and wherein the non-naturally occurring carbonic anhydrase has increased activity at a temperature of greater than about 60 degrees Celsius when compared to the naturally occurring carbonic anhydrase. In an embodiment, the non-naturally occurring carbonic anhydrase has increased activity that is for more than about 30 minutes, 1 hour, 2 hours, 3 hours, 4 hours, 5 hours, 6 hours, 24 hours, 44 hours, 48 hours, and 92 hours. In an embodiment, the non-naturally occurring carbonic anhydrase has increased activity that is at a temperature greater than 65, 70, 75, 80, 85 or 90 degrees Celsius. In an embodiment, the non-naturally occurring carbonic anhydrase has a nucleotide sequence encoding the non-naturally occurring carbonic anhydrase that comprises a sequence that is greater than 70% identical to a sequence selected from the group consisting of SEQ ID NO: 6, SEQ ID NO: 8, SEQ ID NO: 22, SEQ ID NO: 24 and SEQ ID NO: 26. In an embodiment, the non-naturally occurring carbonic anhydrase has an amino acid sequence that is greater than 70% identical to a sequence selected from the group consisting of SEQ ID NO: 5, SEQ ID NO: 7, SEQ ID NO: 21, SEQ ID NO: 23 and SEQ ID NO: 25.

In an aspect, disclosed herein is a method for CO₂ separation and CO₂ capture comprising the step of reacting CO₂ with a non-naturally occurring carbonic anhydrase comprising at least one mutation that results in the substitution of at least one cysteine for at least one amino acid in a naturally occurring carbonic anhydrase; and wherein the non-naturally occurring carbonic anhydrase has increased activity at a temperature of greater than about 60 degrees Celsius when compared to the naturally occurring carbonic anhydrase. In an embodiment, the method contains the step of reacting CO₂ with non-naturally occurring carbonic anhydrase is for more than about 30 minutes, 1 hour, 2 hours, 3 hours, 4 hours, 5 hours, 6 hours, 24 hours, 44 hours, 48 hours, and 92 hours. In an embodiment, the method contains the step of reacting CO 2 with the non-naturally occurring carbonic anhydrase is at a temperature greater than 65, 70, 75, 80, 85 or 90 degrees Celsius. In an embodiment, the the non-naturally occurring carbonic anhydrase comprises a nucleotide sequence that is greater than 70% identical to a sequence selected from the group consisting of SEQ ID NO: 6, SEQ ID NO: 8, SEQ ID NO: 22, SEQ ID NO: 24 and SEQ ID NO: 26. In an embodiment, the non-naturally occurring carbonic anhydrase comprises an amino acid sequence that is greater than 70% identical to a sequence selected from the group consisting of SEQ ID NO: 5, SEQ ID NO: 7, SEQ ID NO: 21, SEQ ID

NO: 23 and SEQ ID NO: 25.

In an aspect, disclosed herein is a system for CO₂ separation and CO₂ capture comprising non-naturally occurring carbonic anhydrases comprising at least one mutation that results in the substitution of at least one cysteine for at least one amino acid in a naturally occurring carbonic anhydrase; and wherein the non-naturally occurring carbonic anhydrase has increased activity at a temperature of greater than about 60 degrees Celsius when compared to the naturally occurring carbonic anhydrase; and wherein the system further comprises a support wherein the with the non-naturally occurring carbonic anhydrases are immobilized to the support; and wherein the non-naturally occurring carbonic anhydrases are contacted with CO₂. In an embodiment, the non-naturally occurring carbonic anhydrase has increased activity for more than about 30 minutes, 1 hour, 2 hours, 3 hours, 4 hours, 5 hours, 6 hours, 24 hours, 44 hours, 48 hours, and 92 hours. In an embodiment, the non-naturally occurring carbonic anhydrases react with CO₂ at a temperature greater than 65, 70, 75, 80, 85 or 90 degrees Celsius. In an embodiment, the CO₂ results from the combustion of fossil fuels or biomass. In an embodiment, the system further comprises a carbon capture unit wherein the carbon capture unit comprises an immobilized biocatalyst comprising an amino acid sequence that is greater than 70% identical to a sequence selected from the group consisting of SEQ ID NO: 5, SEQ ID NO: 7, SEQ ID NO: 21, SEQ ID NO: 23 and SEQ ID NO: 25.

Other objects, advantages, and novel features of the present invention will become apparent from the following detailed description of the invention when considered in conjunction with the accompanying drawings.

### DESCRIPTION OF THE DRAWINGS

FIGS. 1A and 1B depict a dimer of PmaCA. Two protein molecules are shown in cartoon representation and colored grey and green. Blue balls represent zinc ion located at the active sites of each molecule. FIG. 1A, frontal view. FIG. 1B, view from above, rotated 90 degrees.

FIGS. 2A and 2B depict a TaCA tetramer. FIG. 2A—Four protein molecules are shown in cartoon representation and colored as green, magenta, orange, and cyan. Blue balls represent zinc ion located at the active sites of each molecule. FIG. 2B—zoom-in on the disulfide bonds connecting protein molecules in the tetramer in a crisscross fashion.

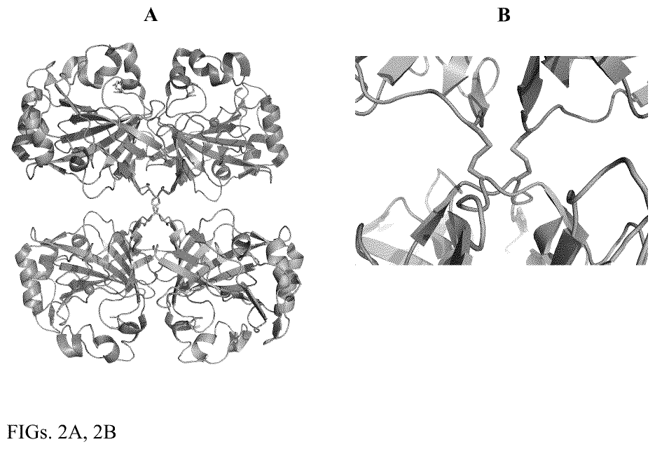

FIG. 3 depicts a superimposition of PmaCA (grey), SspCA (cyan), SazCA (yellow), LogaCA (green), and TaCA (magenta). Proteins are superimposed using one protein chain for each (upper right). The fold is conserved, and the dimerization interface is also conserved.

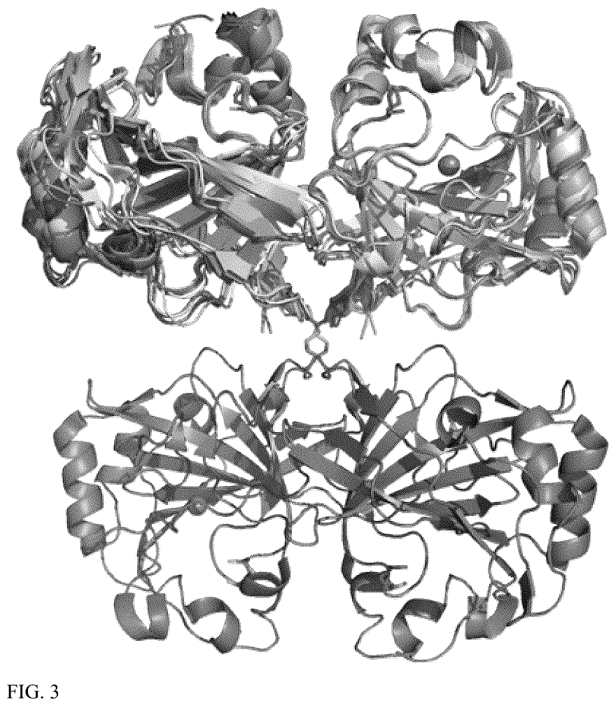

FIG. 4 depicts mutant 1: Gly200Cys, Asn236Cys. View from above. Two disulfides connecting molecules in the dimer are shown as spheres.

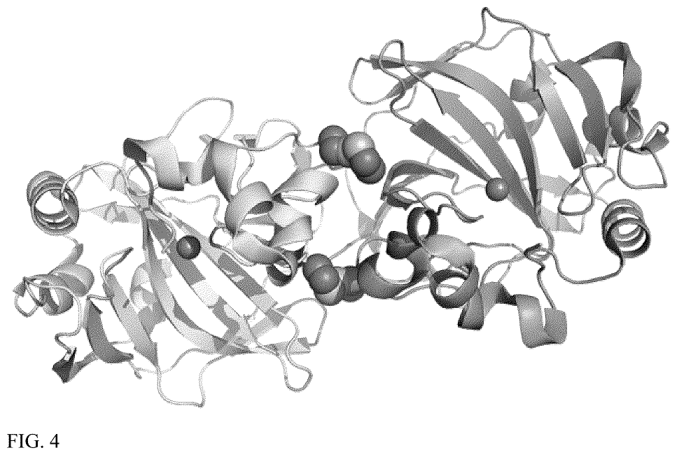

FIG. 5 depicts mutant 2, frontal view: Ala61Cys (makes disulfide with the same Ala61Cys from the 2nd molecule), one disulfide connecting molecules in the dimer is shown as spheres.

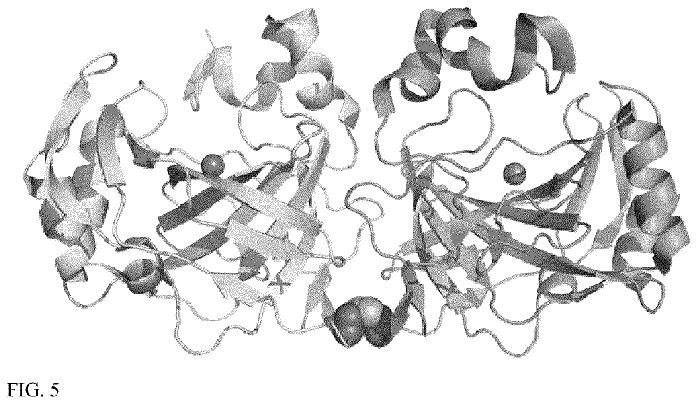

FIG. 6 depicts mutant 3: Ser189Cys, Ala237Cys, view from above. Two disulfides connecting molecules in the dimer are shown as spheres.

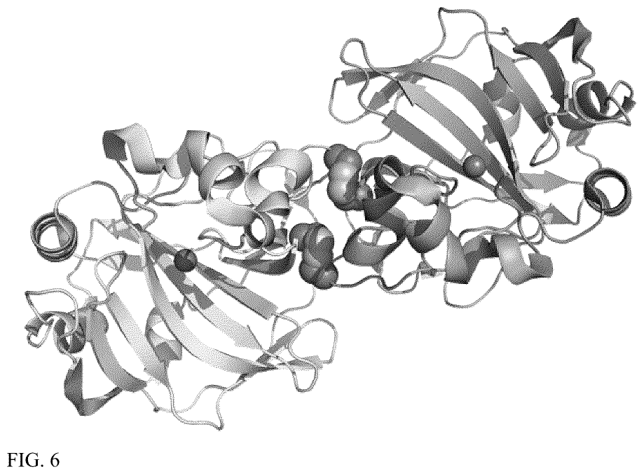

FIG. 7 depicts a sequence alignment for TaCA, PmaCA, LogaCA, SspCA, SazCA and PmaCA mutants 1, 2, and 3. Positions of point mutations are boxed in red for mutant 1, blue for mutant 2, green for mutant 3 indicating where these mutations should be introduced in other CAs.

FIG. 8 depicts activity retained after PmaCA WT and mutants incubation at 60° C. for 2 hours.

FIG. 9 depicts activity retained after PmaCA WT and mutants incubation at 90° C. The proteins were induced and expressed at 35° C. and 45° C. temperatures. Mutants 1, 3, and 2+3 combo did not have high broth activity from the beginning, so the activity of these samples quickly dropped below the method detection range. Despite sharp activity decline in the first hour for the Mutant 2, in the long run (2-48 h range) this mutant retained more activity than the WT.

FIG. 10 depicts activity retained after PmaCA WT and mutants incubation at 90° C. The proteins were induced and expressed at 35° C. and 45° C. temperatures as in Experiment 2. Protein containing broths were concentrated to boost the initial absolute activity numbers. Mutants 1, 3, and 2+3 combo did not have high broth activity from the beginning, so the activity of these samples quickly dropped below the method detection range. At 1 hour mutant 2 already retained higher activity than WT enzyme and in interval 2-72 hours mutant 2 retained significant margin above WT activity. The broth for PmaCA WT enzyme expressed at 45° C. was treated differently from other broths. It was overconcentrated first and then diluted with distilled water to reach the absolute activity levels comparable to other broths.

FIG. 11 depicts activity retained after SazCA WT and mutants 2, 3, and 2+3 combination incubation at 90° C. Proteins were induced and expressed at 45° C. Initial sharp drop of activity within 1st hour could be attributed to the degradation of the portion of a protein that was not able to fold correctly. Since mutant 2 has one single point mutation, mutant 3 has two single point mutation and mutant 2+3 has three single point mutations, without being limited by theory, it is possible that negative effects from the mutations are stacked up and the yield of the properly folded protein decreases with the increase of the number of mutations. After an initial sharp drop, activity is decreasing much slower for the mutants than it is decreasing for the WT enzyme. If we consider the first hour at 90° C. as ‘pre-incubation’ and take activities after 1 hour as 100% for each protein, the retained activity graphs would show the improved stability of the mutants.

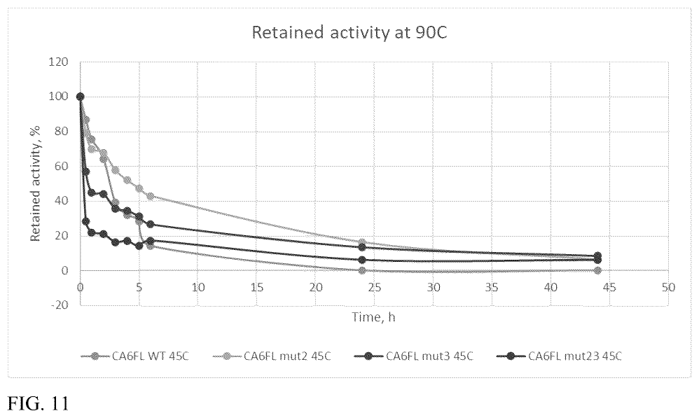

FIG. 12 depicts activity retained after SazCA WT and mutants 2, 3, and 2+3 combination incubation at 90° C. when activity after 1 hour of ‘pre-incubation’ is taken as 100%.

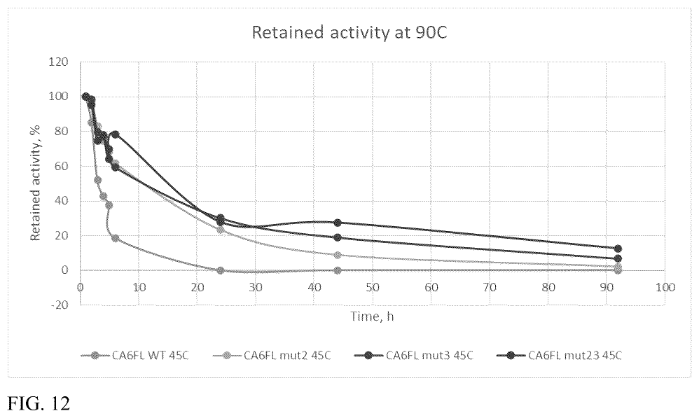

FIG. 13 depicts activity retained after SspCA WT and mutants incubation at 90° C. The proteins were induced and expressed at 35° C. and 45° C. temperatures as in Experiment 2. Protein containing broths were concentrated to boost the initial absolute activity numbers. At five hours, mutant samples were the best performing, retaining about 40% of initial activity.

FIG. 14 depicts SDS-PAGE analysis of CA6FL protein expressed in B. subtilis guided by signal peptide of B. licheniformis alpha-amylase (SPamyL) at 35° C. Lanes 1-3 were the secreted proteins collected at 0, 6 and 12 hours after IPTG induction, respectively. The loading amount is 20 μL (i.e. 15 μL supernatant+5 μL 4×LDS) per well in non-reducing SDS-PAGE. The red box indicated the expression of CA6FL bands.

FIG. 15 depicts fresh broth activity for PmaCA (CA3) WT and mutants induced at 30° C., 35° C., and 45° C. Activity for the mut1, mut3, and mut23 (combination of mutants 2 and 3) was below the measurable threshold for the set induced at 30° C.

FIG. 16 depicts fresh broth activity for LogaCA (CA4) WT and mutants induced at 35° C. and 45° C. Activity for the mut3 and mut23 (combination of mutants 2 and 3) was below the measurable threshold for the set induced at 35° C.

FIG. 17 depicts fresh broth activity for SspCA (CA5) WT and mutants induced at 35° C. and 45° C.

FIG. 18 depicts fresh broth activity for SazCA (CA6) WT and mutants induced at 35° C. and 45° C.

FIG. 19 depicts fresh broth activity for PmaCA (CA3) WT and mutants induced at 45° C. with and without addition of cysteine. Activity for the mut1 was below the measurable threshold for the set induced with or without cysteine. Activity for the mut23 (combination of mutants 2 and 3) was below the measurable threshold for the set induced without cysteine.

FIG. 20 depicts fresh broth activity for PmaCA (CA3) WT and mutants induced at 45° C. with and without addition of cysteine, amino acid mix, or both. Activity for the mut23 (combination of mutants 2 and 3) was below the measurable threshold for the set induced without cysteine.

FIG. 21 depicts fresh broth activity for PmaCA (CA3) WT and mutants induced at 45° C. with and without addition of diamide. Activity for the mut1, and mut23 (combination of mutants 2 and 3) was below the measurable threshold.

### DETAILED DESCRIPTION

Being one of the fastest enzymes known in nature, carbonic anhydrase (CA) catalyzes the interconversion between CO₂ and bicarbonate which accelerates the capture of CO₂ by serving as a catalyst in alkaline capture solvents with slow absorption kinetics. The enzyme accelerated process allows use of more benign and sustainable solvents with low regeneration energy thus reducing energy consumption.

Disclosed herein are CA enzyme candidates with improved catalytic activity, thermostability and solvent compatibility and developed new enzyme immobilization techniques for improving the enzyme longevity and tested more benign and sustainable solvents accelerated by CA for CO₂ capture. The improved enzyme properties together with the novel immobilization technology with selected solvents have the potential to significantly reduce the cost and the energy requirement for CO₂ capture.

Disclosed herein are optimized, highly active and thermostable carbonic anhydrase enzymes, which are needed for testing in a novel and low energy CO₂ scrubbing process. CA is gaining credibility as an efficient catalyst for significantly enhancing reactive CO₂ absorption in low energy solvents. To overcome the high energy requirement of traditional monoethanolamine (MEA)-based CO₂ scrubbing process, disclosed herein are methods, compositions and systems used to develop more efficient CO₂ scrubbing technology by: 1) improving the robustness of CA, including tolerance to high temperature, high solvent concentration and high pH; 2) improving CA longevity using biodegradable enzyme-entrapping polymeric structures (BEEPS); and 3) utilizing environmentally friendly solvents to improve process sustainability.

The most studied CA family currently is alpha-class of CAs with at least five members of the family being characterized biochemically and structurally:
a) Thermovibrio ammonificans—TaCA, CA1
b) Persephonella marina EX-H1—PmaCA, CA3
c) another Persephonella marina CA coming from metagenome sampling at Logachev deep sea vent—LogaCA, CA4
d) Sulfurihydrogenibium yellowstonense YO3AOP1-SspCA, CA5
e) Sulfurihydrogenibium azorense—SazCA, CA6

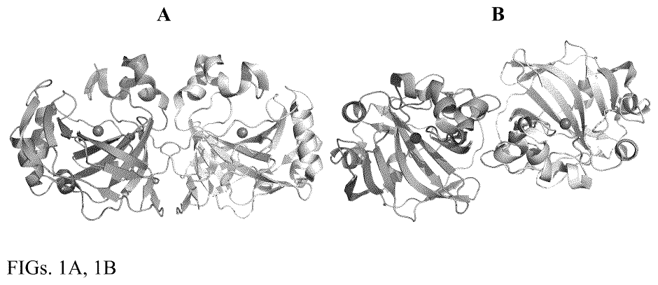

While active site organization of the listed above alpha-class CAs is suited for an independent monomeric function, it seems that all examples (except for TaCA) exist as dimers in the solution, see FIG. 1, for example.

In, for example FIG. 1, the dimerization interface has significant area and is stabilized by hydrophobic interactions, hydrogen bonds and salt bridges. There are no covalent bonds between protein molecules on the dimerization interface. The protein fold is conserved throughout the family and the dimerization interface shares very high similarity among the listed enzymes.

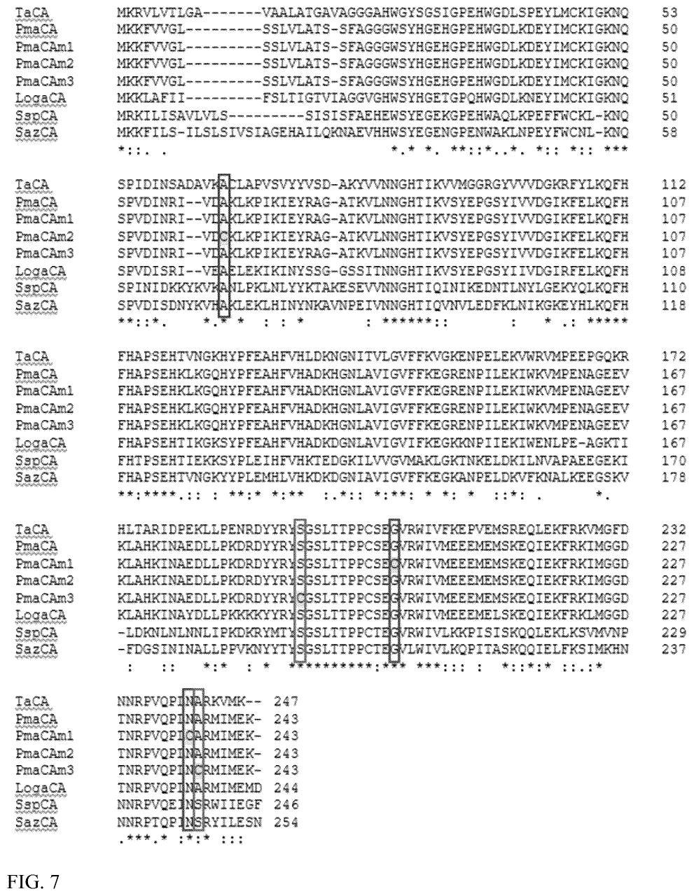

In an embodiment, disclosed herein are novel protein dimers of alpha-CAs via one or more covalent disulfide bonds designed at the dimerization interface via one or more single-point mutations, replacing a native amino acid residue of the enzyme with cysteine. The exact locations of the single-point mutation may be used in alpha-CAs from different species. Three locations for the intermolecular disulfides were designed in the first round, in an embodiment, PmaCA numbering (including signal peptide, SP) is reflected in FIG. 7.

In an embodiment, mutants 1, 2, 3, and 2+3 combination were introduced in PmaCA and 2, 3, and 2+3 in SazCA. For SazCA mutant 1 is Gly210Cys+Asn246Cys (numbering according to the full-length sequence including signal peptide), mutant 2 is Ala71Cys, mutant 3 is Ser199Cys+Ser247Cys. Wild-type (WT) enzymes along with the mutants were expressed in Bacillus subtilis and induced at 35° C. and 45° C. temperatures. Culturing media containing secreted enzymes (broth) was collected, cells were spun down and removed. All enzymes were subjected to the prolonged incubation at 90° C. in form of the broth. Samples were taken out at 30 min, 1 hour, 2 hours, 3 hours, 4 hours, 5 hours, 6 hours, 24 hours, 44-48 hours, and 92 hours. Samples were immediately cooled down to 0° C. and the enzyme activity was tested by Wilbur-Anderson method using colorimetric assay.

Assay description: In an embodiment, an assay is performed on ice at 0° C.-1° C. temperature. All solutions are chilled on ice until the desired temperature is reached. 1 mL of the 20 mM Tris buffer at pH 8.3 was mixed with 0.1 mL pH indicator Bromthymol Blue (BTB). Ten uL (0.01 mL) of broth containing enzyme was added to the mix (nothing added for the control). Then, 1 mL of water fully saturated with CO₂ was added and the stopwatch was started. When BTB changed color from blue to yellow indicating pH dropping below 6.3, stopwatch was stopped. Uncatalyzed reaction time (Tₒ) is longer than catalyzed reaction time (T_{c}) when an activity catalyst is present. Activity in Wilbur-Anderson units is calculated as WAU=(Tₒ−T_{c})/T_{c}.

For the comparison of different enzymes broth activity, this WAU value is then normalized for the dilution factor (DF=Vₜₒₜ/V_{broth} where Vₜₒₜ is a total reaction volume and V_{broth} is the volume of broth added) and optical density of the broth (OD), so the units to compare would be WAU*DF/OD.

For the measurement of retained activity, WAU value of each enzyme at start is taken as 100% for that particular enzyme, and WAU values obtained after various incubation times are compared to the initial WAU activity value.

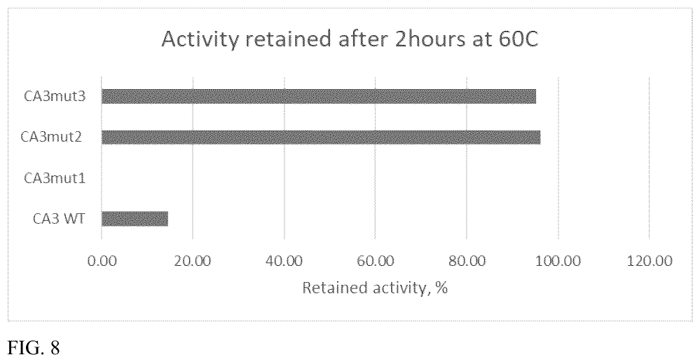

As an example, results for PmaCA enzymes set are depicted in FIG. 8.

To be effective for CO₂ sequestration, CA enzymes need to withstand harsh process conditions, high temperature, high pH, high solvent conditions and tolerance of gas and process contaminants. In an embodiment, the non-naturally occurring CA enzymes disclosed herein 1) improve enzyme robustness including thermotolerance of CA enzymes with fast CO₂ absorption rate, thermostability and solvent compatibility; 2) improve CA longevity using biodegradable enzyme-entrapping polymeric structures (BEEPS); and 3) utilize compatible environmentally friendly solvents to improve process sustainability with lower energy requirement. Thus, disclosed herein are engineered, non-naturally occurring CA enzymes with improved properties including catalytic activity, thermostability and solvent compatibility.

In an embodiment, a large quantity of the improved CA enzyme candidates is needed for fabricating sufficient immobilized biocatalyst materials using enzyme immobilization technology and further testing at the bench-scale integrated carbon capture unit with selected more benign and sustainable solvents with low regeneration energy. In an embodiment, the system (with an internal diameter of 7.6 cm, a packing height of approximately 2 m) was outfitted with instrumentation to allow comprehensive data gathering on temperature profile along the absorber and stripper column to calculate mass transfer flux and regeneration energy consumption, optimize the enzyme production process for scaling-up the production of the improved CA enzyme candidates; and produce up to 100 g of protein for fabricating immobilized biocatalyst and testing at the integrated carbon capture unit. The improved enzyme properties together with the novel immobilization technology with selected solvents provide substantial reduction of the energy requirement and cost for CO₂ capture. In an embodiment, the compositions, methods and systems disclosed herein provide alternative CO₂ capture technologies which can be deployed in many industrial applications for capturing CO₂ from biopower and fossil-based power plants.

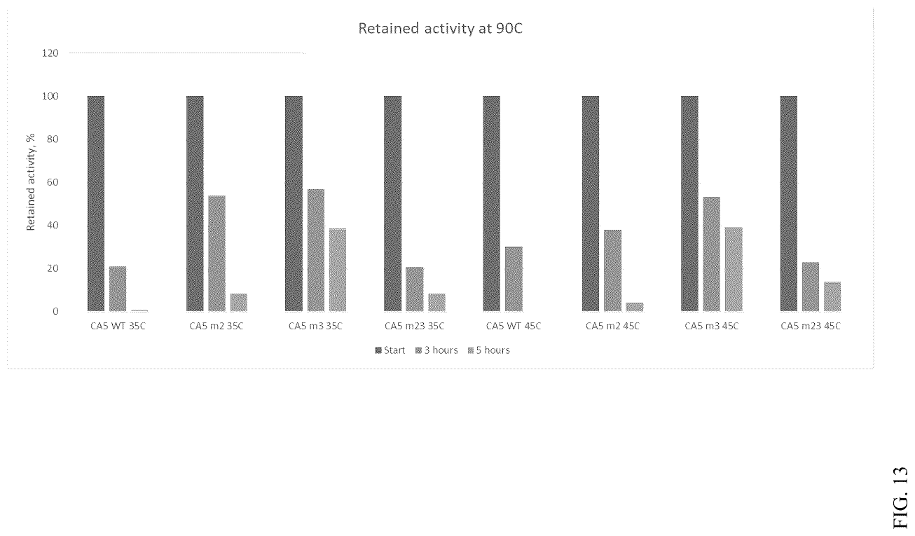

The data graphically depicted in FIG. 13 are also disclosed in Table 1 below:

| | CA5 | CA5 | CA5 | CA5 | CA5 | CA5 | CA5 | CA5 |
|---|---|---|---|---|---|---|---|---|
| Time, | WT | m2 | m3 | m23 | WT | m2 | m3 | m23 |
| h | 35C | 35C | 35C | 35C | 45C | 45C | 45C | 45C |
| 0 | 100 | 100 | 100 | 100 | 100 | 100 | 100 | 100 |
| 3 | 21.1 | 53.8 | 56.8 | 20.6 | 30.2 | 37.9 | 53.3 | 23.0 |
| 5 | 0.8 | 8.5 | 38.5 | 8.3 | 0.0 | 4.4 | 39.2 | 13.9 |

The data depicted in FIG. 8 are also disclosed in Table 2 below:

| CA sample | Activity retained after 2 hours at 60 C. |
|---|---|
| CA3 WT | 14.61 |
| CA3mut1 | 0.00 |
| CA3mut2 | 96.24 |
| CA3mut3 | 95.32 |

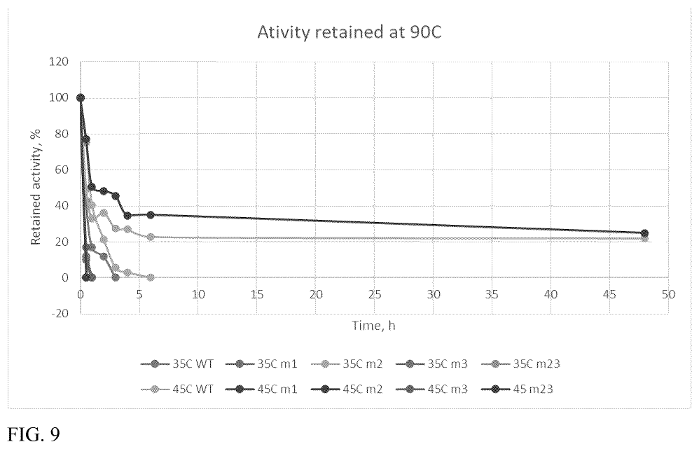

The data depicted in FIG. 9 are also disclosed in Table 3 below:

| time, h | 35C WT | 35C m1 | 35C m2 | 35C m3 | 35C m23 | 45C WT | 45C m1 | 45C m2 | 45C m3 | 45 m23 |
|---|---|---|---|---|---|---|---|---|---|---|
| 0 | 100 | 100 | 100 | 100 | 100 | 100 | 100 | 100 | 100 | 100 |
| 0.5 | 42.50 | 10.08 | 49.41 | 11.78 | 0.00 | 75.11 | 0.00 | 76.70 | 16.92 | 0.00 |
| 1 | 16.89 | 0 | 32.91 | 0 | | 40.28 | | 50.40884 | 0.00 | |
| 2 | 11.89 | | 36.11 | | | 21.24 | | 48.00 | | |
| 3 | 0.00 | | 27.25 | | | 5.48 | | 45.31 | | |
| 4 | | | 27.05 | | | 3.04 | | 34.39 | | |
| 6 | | | 22.77 | | | 0.00 | | 34.80 | | |
| 48 | | | 21.95 | | | | | 24.72 | | |

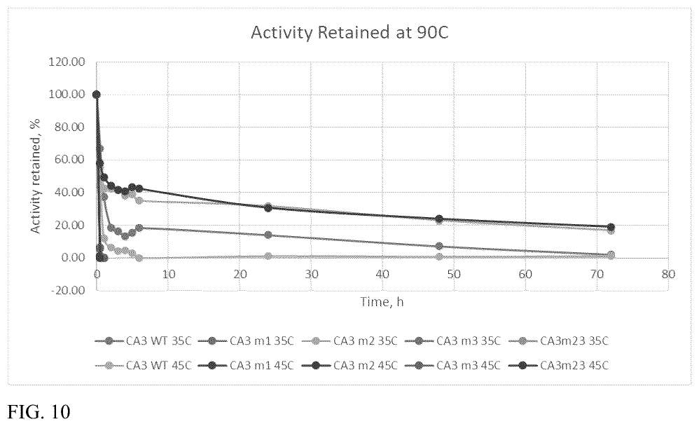

The data depicted in FIG. 10 are also disclosed in Table 4 below:

| Time, | CA3 WT | CA3 m1 | CA3 m2 | CA3 m3 | CA3m23 | CA3 WT | CA3 m1 | CA3 m2 | CA3 m3 | CA3m23 |
|---|---|---|---|---|---|---|---|---|---|---|
| h | 35C | 3 [symbol] | 3 [symbol] | 3 [symbol] | 3 | 4 [symbol] | 4 [symbol] | 4 [symbol] | 4 [symbol] | 45C |
| 0 | 100.00 | 100.00 | 100.00 | 100.00 | 100.00 | 100.00 | 100.00 | 100.00 | 100.00 | 100.00 |
| 0.5 | 66.70 | 6.26 | 55.26 | 6.09 | 0.00 | 44.08 | 1.14 | 57.99 | 5.37 | 0.00 |
| 1 | 37.24 | 0.00 | 42.48 | 0.00 | | 11.79 | 0.00 | 49.40 | 0.00 | |
| 2 | 18.38 | | 42.42 | | | 6.05 | | 43.92 | | |
| 3 | 16.30 | | 41.67 | | | 3.94 | | 41.39 | | |
| 4 | 12.93 | | 37.84 | | | 4.52 | | 40.45 | | |
| 5 | 15.35 | | 38.74 | | | 2.93 | | 43.06 | | |
| 6 | 18.18 | | 34.87 | | | 0.00 | | 42.14 | | |
| 24 | 13.85 | | 31.56 | | | 1.09 | | 30.51 | | |
| 48 | 6.98 | | 22.80 | | | 0.70 | | 23.75 | | |
| 72 | 1.89 | | 16.64 | | | 0.94 | | 18.85 | | |

[symbol]

indicates data missing or illegible when filed

### EXPERIMENTAL EXAMPLES

### B. subtilis Strain and the Preparation of Competent Cells

B. subtilis strain WB800N strain was obtained from MoBiTec GmbH (Gottingen, Germany), and used as the host strain for extracellular expression of CAs. WB800N strain was an eightfold extracellular protease deficient derivative of strain 168, with genotype of nprE aprE epr bpr mpr::ble nprB::bsr Δvpr wprA::hyg cm::neo; NeoR (i.e. carries resistance to neomycin). The competent cells of WB800N were prepared according to the technical guide provided by the above company.

### Expression Vector, the Design of the Constructs for Expressing CAs in B. subtilis

Bacillus expression vector pHT43 was obtained from MoBiTec GmbH (Gottingen, Germany).

### Signal Peptides, Gene Synthesis and Subcloning into Vector to Build the Constructs for Expressing CAs in B. subtilis

The signal peptide of Bacillus licheniformis alpha-amylase (i.e. AmyL; uniprot ID, P06278) is a 29 aa signal peptide named as SPamyL, MKQQKRLYARLLTLLFALIFLLPHSAAAA (SEQ ID NO: 35); this signal peptide was used for the expression and secretion of CAs.

The sequence of each CA gene was codon-optimized using B. subtilis codon usage frequency and synthesized by GenScript Inc (Piscataway, New Jersey); it had KpnI site at 5′ end, and stop codon-XbaI (taatctaga) at 3′ end, and was composed of 87 nucleotides coding for the 29 aa of signal peptide SPamyL, followed by the codon-optimized CA gene sequence, as disclosed herein in SEQ ID NOs 2, 4, 6, 8, 10, 12, 14, 16, 18, 20, 22, 24, 26, 28, 30, 32, and 34.

For the subcloning, digest the above synthesized gene with KpnI-XbaI, and linked into KpnI-XbaI cut pHT43 vector. The obtained plasmids were used for transformation as described below.

### Transformation and Engineered Strains

These above plasmids plus the empty vector pTH43 were transformed into B. subtilis WB800N competent cells, using the procedure according to the technical guide provided by the above company. The obtained strains were listed in the below table.

TABLE 5
Plasmids and strains for expressing carbonic anhydrase (CA) enzymes
in B. subtilis using WB800N (shorten as strain 800) as host cell.

| | Plasmids with signal peptide and | Amino Acid | DNA |
|---|---|---|---|
| Strains | CA description | SEQ ID NO. | SEQ ID NO. |
| 800-1-EV | pHT43 EV | | |
| 800-CA3 | pHT43-SPamyL-PmaCA-CA3 | SEQ ID NO: 1 | SEQ ID NO: 2 |
| 800-CA3mut1 | pHT43-SPamyL-PmaCA-CA3mut1 | SEQ ID NO: 3 | SEQ ID NO: 4 |
| | with G181C and N217C | | |
| 800-CA3mut2 | pHT43-SPamyL-PmaCA-CA3mut2 | SEQ ID NO: 5 | SEQ ID NO: 6 |
| | with A42C | | |
| 800-CA3mut3 | pHT43-SPamyL-PmaCA-CA3mut3 | SEQ ID NO: 7 | SEQ ID NO: 8 |
| | with S170C and N217C | | |
| 800-CA3mut23 | pHT43-SPamyL-PmaCA-CA3mut23 | SEQ ID NO: 9 | SEQ ID NO: 10 |
| 800-CA4 | pHT43-SPamyL-LOGACA-CA4 | SEQ ID NO: 11 | SEQ ID NO: 12 |
| 800-CA4mut2 | pHT43-SPamyL-LOGACA-CA4mut2 | SEQ ID NO: 13 | SEQ ID NO: 14 |
| 800-CA4mut3 | pHT43-SPamyL-LOGACA-CA4mut3 | SEQ ID NO: 15 | SEQ ID NO: 16 |
| 800-CA4mut23 | pHT43-SPamyL-LOGACA-CA4mut23 | SEQ ID NO: 17 | SEQ ID NO: 18 |
| 800-CA5 | pHT43-SPamyL-SspCA-CA5 | SEQ ID NO: 19 | SEQ ID NO: 20 |
| 800-CA5mut2 | pHT43-SPamyL-SspCA-CA5mut2 | SEQ ID NO: 21 | SEQ ID NO: 22 |
| 800-CA5mut3 | pHT43-SPamyL-SspCA-CA5mut3 | SEQ ID NO: 23 | SEQ ID NO: 24 |
| 800-CA5mut23 | pHT43-SPamyL-SspCA-CA5mut23 | SEQ ID NO: 25 | SEQ ID NO: 26 |
| 800-CA6FL | pHT43-SPamyL-fullSazCA | SEQ ID NO: 27 | SEQ ID NO: 28 |
| 800-CA6FLmut2 | pHT43-SPamyL-fullSazCAmut2 | SEQ ID NO: 29 | SEQ ID NO: 30 |
| 800-CA6FLmut3 | pHT43-SPamyL-fullSazCAmut3 | SEQ ID NO: 31 | SEQ ID NO: 32 |
| 800-CA6FLmut23 | pHT43-SPamyL-fullSazCAmut23 | SEQ ID NO: 33 | SEQ ID NO: 34 |

### Expression and Secretion of CA Proteins Induced with IPTG at the Default 35° C.

Since the plasmids we built contain signal peptide SPamyL, the recombinant CAs were expected to be secreted into the medium. To test the secretion of CAs, obtained transformants were cultured. Briefly, inoculate the recombinant B. subtilis strains from plate or glycerol storage into 5 mL fresh 2xYT medium (16 g/L tryptone, 10 g/L yeast extract, 5 g/L NaCl, final pH 7.0) supplemented with neomycin 10 μg/mL and chloramphenicol (5 μg/mL), and cultured in a shaker at 35° C., 210 rpm. Inoculated the above seed culture into 20 mL fresh 2xYT medium supplemented with neomycin 10 μg/mL and chloramphenicol (5 μg/mL) in a 125-mL flask to an OD₆₀₀ of 0.15. The cultures were grown in a shaker at 35° C., 210 rpm until the OD₆₀₀ reached 0.7-0.8, then it was induced with 1 mM IPTG and 0.5 mM ZnSO₄, by which two aliquots of samples being collected and defined as T₀: 100 uL and 1 mL). The cultures were continued to grow in a shaker at 35° C. and 130 rpm.

Similarly, three aliquots were collected at 6 and 12 h after the induction (defined as T₆ and T₁₂ samples): 100 μL and 1 mL. These T₀ and T₁₂ samples were centrifuged at 12,000 rpm, 10 min, 4° C. to separate the supernatants and pellets. While 100 μL supernatant was mixed with 33 μL 4×LDS sample buffer, the pellets from 100 uL culture were suspended in 133 μL 1×LDS sample buffer; both being heated at 95° C. for 5 min, followed by centrifugation at 12,000 rpm for 2 min to remove any debris. For these protein samples, 20 μL of each preparation was analyzed with SDS-PAGE.

### Expression and Secretion of CA Proteins Induced with IPTG at 30, 35 and 45° C.

The expression and secretion of CA proteins by the mutants were also examined after being induced at 30, 35 and 45° C. for 12 hours. The procedures for seed culture preparation, the inoculation into the fresh 2XYT and the initial culturing to OD₆₀₀ of 0.7-0.8 at the default 35° C. were the same as described in the above section of “Expression and secretion of CA proteins induced at the default 35° C.”. When the OD₆₀₀ reached 0.7-0.8, 1 mM IPTG and 0.5 mM ZnSO₄ (final concentration) were added into 20 mL culture in 125-mL flasks. The flasks were transferred to different shakers set at designated either 30° C., or 35° C., or 45° C., with a speed of 130 rpm for 12 hours. The samples were harvested and centrifuged as described above, with the supernatants being collected and stored at 4° C. until being analyzed for the CA activity, heat treatment and thermostability analyses.

### Expression and Secretion of CA Proteins Induced with IPTG with the Supplements of Cysteine or Diamide

To test if the supplements of cysteine or diamide has any impacts on the folding, secretion and functionality of CA proteins, the expression and secretion of CA proteins by the mutants were investigated after being induced at OD₆₀₀ of 0.7-0.8 with a mixture of IPTG and ZnSO₄ without (as the control) or with cysteine or diamide, using the final concentrations as listed below, followed by continuing shaking at 130 rpm at designated 30° C., or 35° C., or 45° C. for 12 hours.

Treatments with cysteine or diamide supplements (with final concentration added into the medium at OD₆₀₀ of 0.7-0.8):
(1). Control treatment: 1 mM IPTG+0.5 mM ZnSO₄
(2). Cysteine treatment: 1 mM IPTG+0.5 mM ZnSO₄+4 mM cysteine
(3). Diamide treatment: 1 mM IPTG+0.5 mM ZnSO₄+0.25 mM diamide

By default, the cysteine stock was freshly prepared unless it was indicated otherwise. The diamide stock also was also freshly prepared.

### Expression and Secretion of CA6FL as a Representative CA

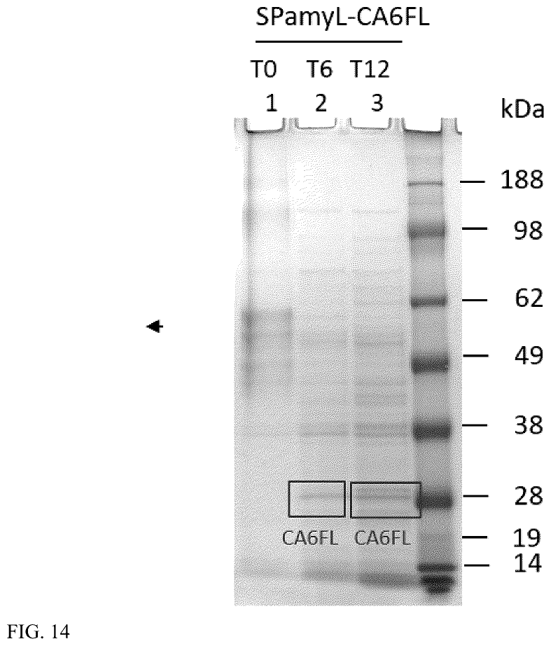

SDS-PAGE analysis of the cell supernatants reveal that we have successfully expressed CA6FL as a representative CA with signal peptide SPamyL, Furthermore, 6 h of IPTG induction at 35° C. is sufficient to lead CA6FL expression and secretion at substantial levels, while a longer IPTG induction time to 12 hours led to higher expression and secretion levels of CA6FL (FIG. 14). Thus, 12 h of IPTG induction at 35° C. was more desirable than 6 h IPTG induction.

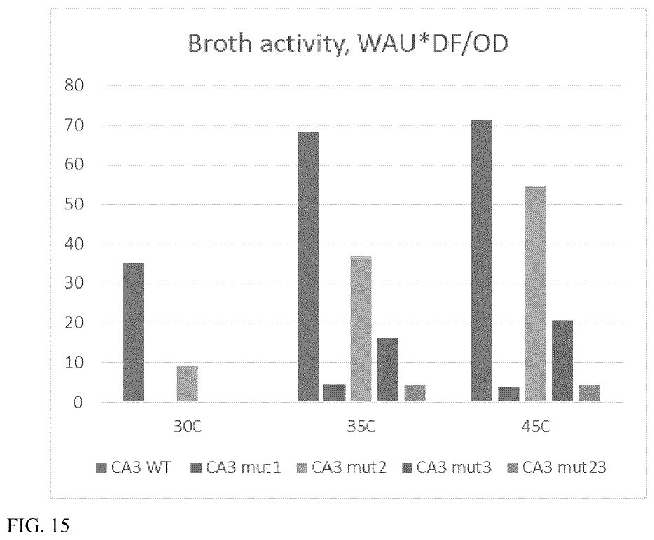

In an embodiment, elevated temperature leads to better CA expression in B. subtilis. Experiment 1: Wild-type (WT) enzymes along with the mutants were expressed in Bacillus subtilis and induced at 30° C., 35° C., and 45° C. for PmaCA (CA3), see FIG. 15.

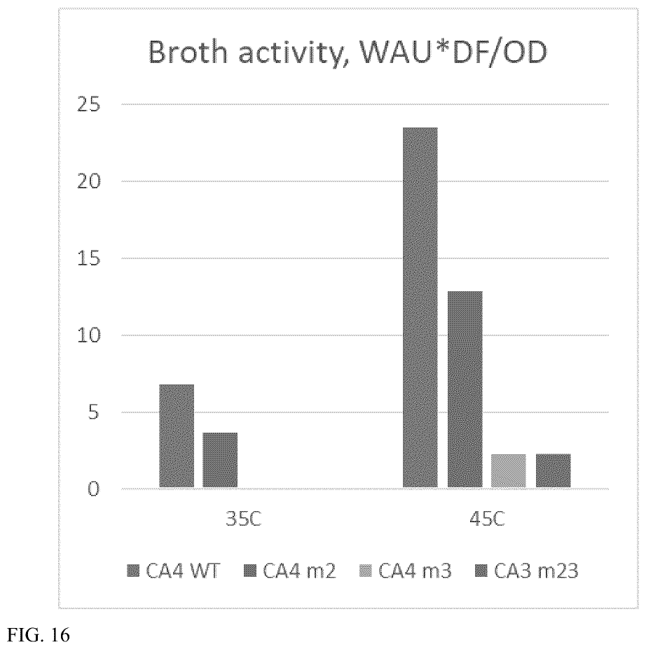

Experiment 2: Wild-type (WT) enzymes along with the mutants were expressed in Bacillus subtilis and induced at 35° C., and 45° C. for LogaCA, see FIG. 16.

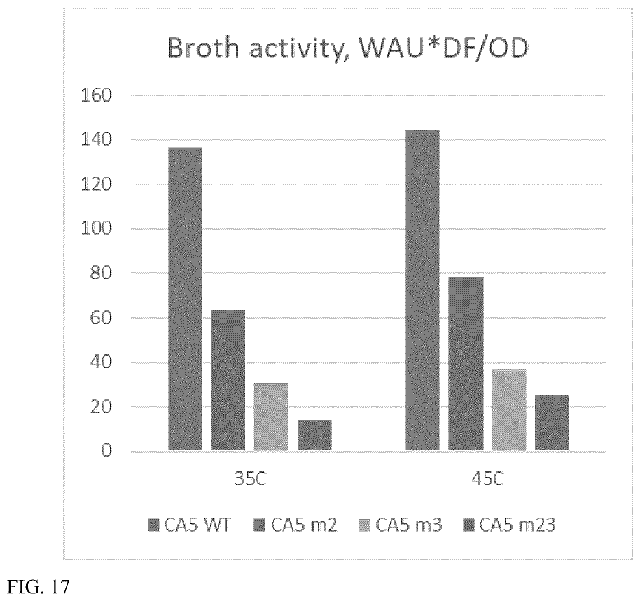

Experiment 3: Wild-type (WT) enzymes along with the mutants were expressed in Bacillus subtilis and induced at 35° C., and 45° C. for SspCA (CA5), see FIG. 17.

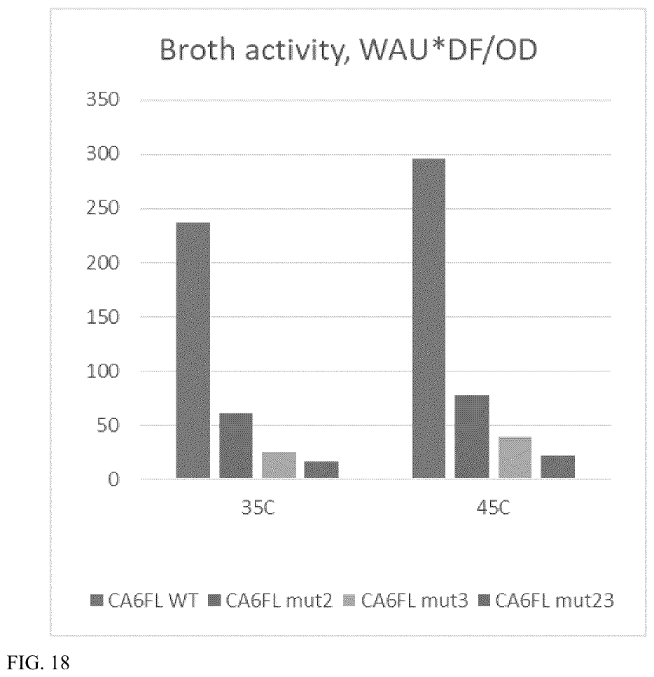

Experiment 4: Wild-type (WT) enzymes along with the mutants were expressed in Bacillus subtilis and induced at 35° C., and 45° C. for SazCA (CA6), see FIG. 18.

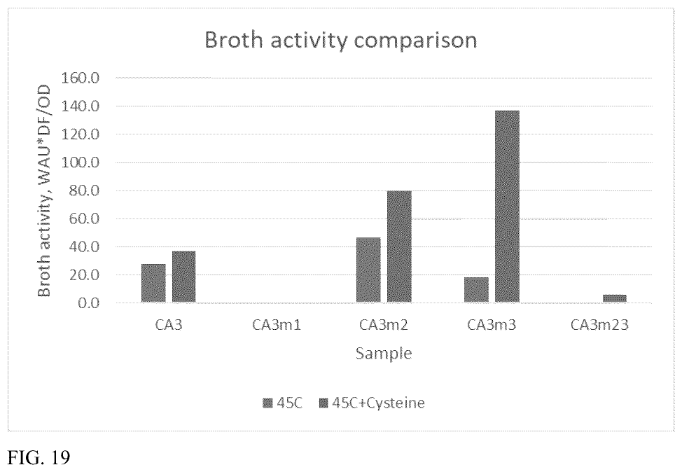

In an embodiment, addition of free cysteine to the expression media leads to the better CA expression. Experiment 5: Wild-type (WT) enzymes along with the mutants were expressed in Bacillus subtilis and induced at 45° C. for PmaCA (CA3) with and without addition of the cysteine, see FIG. 19.

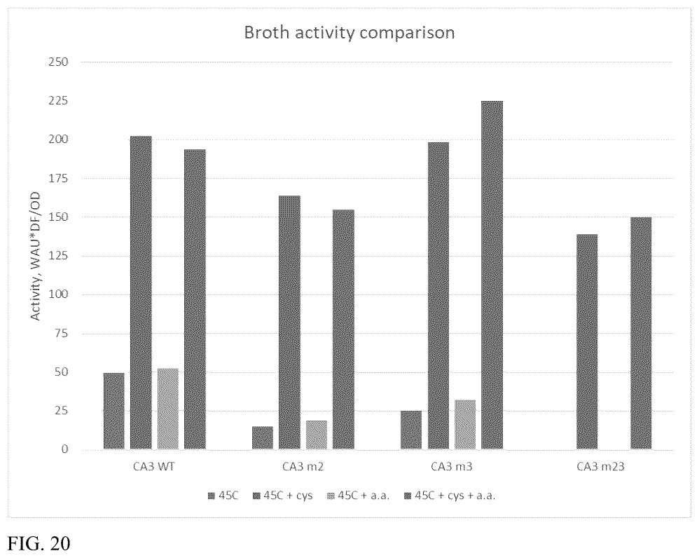

Experiment 6: Wild-type (WT) enzymes along with the mutants were expressed in Bacillus subtilis and induced at 45° C. for PmaCA (CA3) with and without addition of the cysteine, see FIG. 20.

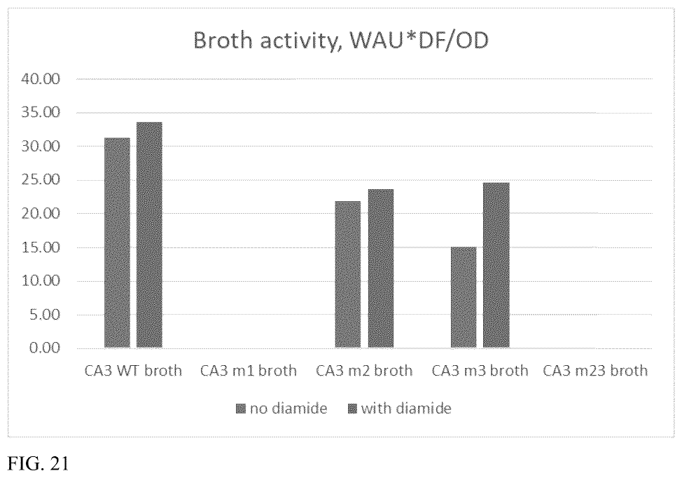

In an embodiment, addition of diamide to the expression media leads to the improved CA expression. Experiment 7: Wild-type (WT) enzymes along with the mutants were expressed in Bacillus subtilis and induced at 45° C. for PmaCA (CA3) with and without addition of the diamide, see FIG. 21.

The following sequences are embodiments of amino acid and nucleotide sequences representing the genes encoding for engineered CAs disclosed herein.

SEQ ID NO:1 and SEQ ID NO: 2

SEQ name: SPamyL-PmaCA-CA3

LENGTH: 253 for PRT; 762 for DNA

TYPE: PRT; DNA

ORGANISM: Signal peptide SPamyL from Bacillus licheniformis; PmaCA-CA3 PRT from Persephonella marina. Synthetic for DNA

(PRT)
SEQ ID NO: 1
MKQQKRLYARLLTLLFALIFLLPHSAAAAGGGWSYHGEHGPEHWGDLKD
EYIMCKIGKNQSPVDINRIVDAKLKPIKIEYRAGATKVLNNGHTIKVSY
EPGSYIVVDGIKFELKQFHFHAPSEHKLKGQHYPFEAHFVHADKHGNLA
VIGVFFKEGRENPILEKIWKVMPENAGEEVKLAHKINAEDLLPKDRDYY
RYSGSLTTPPCSEGVRWIVMEEEMEMSKEQIEKFRKIMGGDTNRPVQPL
NARMIMEK
(DNA)
SEQ ID NO: 2
ATGAAACAACAGAAAAGACTGTATGCACGCCTGCTTACATTACTGTTTG
CTCTTATTTTTCTTTTACCGCATTCAGCAGCGGCTGCCGGCGGAGGATG
GAGCTATCATGGCGAACATGGACCTGAACATTGGGGTGACCTGAAAGAC
GAATATATTATGTGCAAAATCGGCAAAAATCAATCACCGGTTGATATTA
ACAGAATCGTGGATGCAAAACTTAAACCGATCAAAATCGAATATCGCGC
AGGAGCGACAAAAGTCCTGAACAACGGCCATACAATCAAAGTTTCTTAT
GAACCGGGATCATATATTGTTGTGGATGGCATCAAATTTGAATTAAAAC
AATTTCATTTTCATGCACCGAGCGAACATAAACTGAAAGGACAGCATTA
TCCGTTTGAAGCTCATTTTGTTCATGCCGATAAACATGGCAATCTGGCT
GTCATCGGAGTTTTCTTTAAAGAAGGCAGAGAAAACCCGATTCTTGAAA
AAATCTGGAAAGTGATGCCGGAAAATGCCGGCGAAGAAGTCAAATTAGC
ACATAAAATCAACGCGGAAGATTTACTGCCGAAAGATAGAGATTATTAT
CGCTATTCAGGAAGCCTGACAACACCGCCGTGCAGCGAAGGCGTGAGAT
GGATCGTCATGGAAGAAGAAATGGAAATGTCTAAAGAACAGATCGAAAA
ATTTCGCAAAATCATGGGAGGCGATACGAACCGTCCTGTGCAGCCGTTG
AATGCGAGAATGATTATGGAAAAATAA

SEQ ID NO:3 and SEQ ID NO: 4

SEQ name: SPamyL-PmaCA-CA3mut1

LENGTH: 253 for PRT; 762 for DNA

TYPE: PRT; DNA

ORGANISM: Signal peptide SPamyL from Bacillus licheniformis; PmaCA-CA3mut1 modified from Persephonella marina.

(PRT)
SEQ ID NO: 3
MKQQKRLYARLLTLLFALIFLLPHSAAAAGGGWSYHGEHGPEHWGDLKD
EYIMCKIGKNQSPVDINRIVDAKLKPIKIEYRAGATKVLNNGHTIKVSY
EPGSYIVVDGIKFELKQFHFHAPSEHKLKGQHYPFEAHFVHADKHGNLA
VIGVFFKEGRENPILEKIWKVMPENAGEEVKLAHKINAEDLLPKDRDYY
RYSGSLTTPPCSECVRWIVMEEEMEMSKEQIEKFRKIMGGDTNRPVQPL
CARMIMEK
(DNA)
SEQ ID NO: 4
ATGAAACAACAGAAAAGACTGTATGCACGCCTGCTTACATTACTGTTTG
CTCTTATTTTTCTTTTACCGCATTCAGCAGCGGCTGCCGGCGGAGGATG
GAGCTATCATGGCGAACATGGACCTGAACATTGGGGTGACCTGAAAGAC
GAATATATTATGTGCAAAATCGGCAAAAATCAATCACCGGTTGATATTA
ACAGAATCGTGGATGCAAAACTTAAACCGATCAAAATCGAATATCGCGC
AGGAGCGACAAAAGTCCTGAACAACGGCCATACAATCAAAGTTTCTTAT
GAACCGGGATCATATATTGTTGTGGATGGCATCAAATTTGAATTAAAAC
AATTTCATTTTCATGCACCGAGCGAACATAAACTGAAAGGACAGCATTA
TCCGTTTGAAGCTCATTTTGTTCATGCCGATAAACATGGCAATCTGGCT
GTCATCGGAGTTTTCTTTAAAGAAGGCAGAGAAAACCCGATTCTTGAAA
AAATCTGGAAAGTGATGCCGGAAAATGCCGGCGAAGAAGTCAAATTAGC
ACATAAAATCAACGCGGAAGATTTACTGCCGAAAGATAGAGATTATTAT
CGCTATTCAGGAAGCCTGACAACACCGCCGTGCAGCGAATGCGTGAGAT
GGATCGTCATGGAAGAAGAAATGGAAATGTCTAAAGAACAGATCGAAAA
ATTTCGCAAAATCATGGGAGGCGATACGAACCGTCCTGTGCAGCCGTTG
TGTGCGAGAATGATTATGGAAAAATAA

SEQ ID NO: 5 and SEQ ID NO: 6

SEQ name: SPamyL-PmaCA-CA3mut2

LENGTH: 253 for PRT; 762 for DNA

TYPE: PRT; DNA

ORGANISM: Signal peptide SPamyL from Bacillus licheniformis; PmaCA-CA3mut2 modified from Persephonella marina.

(PRT)
SEQ ID NO: 5
MKQQKRLYARLLTLLFALIFLLPHSAAAAGGGWSYHGEHGPEHWG
DLKDEYIMCKIGKNQSPVDINRIVDCKLKPIKIEYRAGATKVLN
NGHTIKVSYEPGSYIVVDGIKFELKQFHFHAPSEHKLKGQHYPFE
AHFVHADKHGNLAVIGVFFKEGRENPILEKIWKVMPENAGEEVKL
AHKINAEDLLPKDRDYYRYSGSLTTPPCSEGVRWIVMEEEMEMSK
EQIEKFRKIMGGDTNRPVQPLNARMIMEK
(DNA)
SEQ ID NO: 6
ATGAAACAACAGAAAAGACTGTATGCACGCCTGCTTACATTACTG
TTTGCTCTTATTTTTCTTTTACCGCATTCAGCAGCGGCTGCCGG
CGGAGGATGGAGCTATCATGGCGAACATGGACCTGAACATTGGGG
TGACCTGAAAGACGAATATATTATGTGCAAAATCGGCAAAAATCA
ATCACCGGTTGATATTAACAGAATCGTGGATTGTAAACTTAAACC
GATCAAAATCGAATATCGCGCAGGAGCGACAAAAGTCCTGAACAA
CGGCCATACAATCAAAGTTTCTTATGAACCGGGATCATATATTGT
TGTGGATGGCATCAAATTTGAATTAAAACAATTTCATTTTCATGC
ACCGAGCGAACATAAACTGAAAGGACAGCATTATCCGTTTGAAGC
TCATTTTGTTCATGCCGATAAACATGGCAATCTGGCTGTCATCGG
AGTTTTCTTTAAAGAAGGCAGAGAAAACCCGATTCTTGAAAAAAT
CTGGAAAGTGATGCCGGAAAATGCCGGCGAAGAAGTCAAATTAGC
ACATAAAATCAACGCGGAAGATTTACTGCCGAAAGATAGAGATTA
TTATCGCTATTCAGGAAGCCTGACAACACCGCCGTGCAGCGAAGG
CGTGAGATGGATCGTCATGGAAGAAGAAATGGAAATGTCTAAAGA
ACAGATCGAAAAATTTCGCAAAATCATGGGAGGCGATACGAACCG
TCCTGTGCAGCCGTTGAATGCGAGAATGATTATGGAAAAATAA

SEQ ID NO: 7 and SEQ ID NO: 8

SEQ name: SPamyL-PmaCA-CA3mut3

LENGTH: 253 for PRT; 762 for DNA

TYPE: PRT; DNA

ORGANISM: Signal peptide SPamyL from Bacillus licheniformis; PmaCA-CA3mut3 modified from Persephonella marina.

(PRT)
SEQ ID NO: 7
MKQQKRLYARLLTLLFALIFLLPHSAAAAGGGWSYHGEHGPEHWG
DLKDEYIMCKIGKNQSPVDINRIVDAKLKPIKIEYRAGATKVLN
NGHTIKVSYEPGSYIVVDGIKFELKQFHFHAPSEHKLKGQHYPFE
AHFVHADKHGNLAVIGVFFKEGRENPILEKIWKVMPENAGEEVKL
AHKINAEDLLPKDRDYYRYCGSLTTPPCSEGVRWIVMEEEMEMSK
EQIEKFRKIMGGDTNRPVQPLCARMIMEK
(DNA)
SEQ ID NO: 8
ATGAAACAACAGAAAAGACTGTATGCACGCCTGCTTACATTACTG
TTTGCTCTTATTTTTCTTTTACCGCATTCAGCAGCGGCTGCCGG
CGGAGGATGGAGCTATCATGGCGAACATGGACCTGAACATTGGGG
TGACCTGAAAGACGAATATATTATGTGCAAAATCGGCAAAAATCA
ATCACCGGTTGATATTAACAGAATCGTGGATGCAAAACTTAAACC
GATCAAAATCGAATATCGCGCAGGAGCGACAAAAGTCCTGAACAA
CGGCCATACAATCAAAGTTTCTTATGAACCGGGATCATATATTGT
TGTGGATGGCATCAAATTTGAATTAAAACAATTTCATTTTCATGC
ACCGAGCGAACATAAACTGAAAGGACAGCATTATCCGTTTGAAGC
TCATTTTGTTCATGCCGATAAACATGGCAATCTGGCTGTCATCGG
AGTTTTCTTTAAAGAAGGCAGAGAAAACCCGATTCTTGAAAAAAT
CTGGAAAGTGATGCCGGAAAATGCCGGCGAAGAAGTCAAATTAGC
ACATAAAATCAACGCGGAAGATTTACTGCCGAAAGATAGAGATTA
TTATCGCTATTGTGGAAGCCTGACAACACCGCCGTGCAGCGAAGG
CGTGAGATGGATCGTCATGGAAGAAGAAATGGAAATGTCTAAAGA
ACAGATCGAAAAATTTCGCAAAATCATGGGAGGCGATACGAACCG
TCCTGTGCAGCCGTTGTGTGCGAGAATGATTATGGAAAAATAA

SEQ ID NO: 9 and SEQ ID NO: 10

SEQ name: SPamyL-PmaCA-CA3mut23

LENGTH: 253 for PRT; 762 for DNA

TYPE: PRT; DNA

ORGANISM: Signal peptide SPamyL from Bacillus licheniformis; PmaCA-CA3mut23 modified from Persephonella marina.

(PRT)
SEQ ID NO: 9
MKQQKRLYARLLTLLFALIFLLPHSAAAAGGGWSYHGEHGPEHWG
DLKDEYIMCKIGKNQSPVDINRIVDCKLKPIKIEYRAGATKVLN
NGHTIKVSYEPGSYIVVDGIKFELKQFHFHAPSEHKLKGQHYPFE
AHFVHADKHGNLAVIGVFFKEGRENPILEKIWKVMPENAGEEVKL
AHKINAEDLLPKDRDYYRYCGSLTTPPCSEGVRWIVMEEEMEMSK
EQIEKFRKIMGGDTNRPVQPLNCRMIMEK
(DNA)
SEQ ID NO: 10
ATGAAACAACAGAAAAGACTGTATGCACGCCTGCTTACATTACTG
TTTGCTCTTATTTTTCTTTTACCGCATTCAGCAGCGGCTGCCGG
CGGAGGATGGAGCTATCATGGCGAACATGGACCTGAACATTGGGG
TGACCTGAAAGACGAATATATTATGTGCAAAATCGGCAAAAATCA
ATCACCGGTTGATATTAACAGAATCGTGGATTGTAAACTTAAACC
GATCAAAATCGAATATCGCGCAGGAGCGACAAAAGTCCTGAACAA
CGGCCATACAATCAAAGTTTCTTATGAACCGGGATCATATATTGT
TGTGGATGGCATCAAATTTGAATTAAAACAATTTCATTTTCATGC
ACCGAGCGAACATAAACTGAAAGGACAGCATTATCCGTTTGAAGC
TCATTTTGTTCATGCCGATAAACATGGCAATCTGGCTGTCATCGG
AGTTTTCTTTAAAGAAGGCAGAGAAAACCCGATTCTTGAAAAAAT
CTGGAAAGTGATGCCGGAAAATGCCGGCGAAGAAGTCAAATTAGC
ACATAAAATCAACGCGGAAGATTTACTGCCGAAAGATAGAGATTA
TTATCGCTATTGTGGAAGCCTGACAACACCGCCGTGCAGCGAAGG
CGTGAGATGGATCGTCATGGAAGAAGAAATGGAAATGTCTAAAGA
ACAGATCGAAAAATTTCGCAAAATCATGGGAGGCGATACGAACCG
TCCTGTGCAGCCGTTGTGTGCGAGAATGATTATGGAAAAATAA

SEQ ID NO: 11 and SEQ ID NO: 12

SEQ name: SPamyL-LOGACA-CA4

LENGTH: 255 for PRT; 768 for DNA

ORGANISM: Signal peptide SPamyL from Bacillus licheniformis; LOGACA-CA4 from deep sea thermal vent.

(PRT)
SEQ ID NO: 11
MKQQKRLYARLLTLLFALIFLLPHSAAAAGGVGHWSYHGETGPQH
WGDLKNEYIMCKIGKNQSPVDISRIVEAELEKIKINYSSGGSSI
TNNGHTIKVSYEPGSYIIVDGIRFELKQFHFHAPSEHTIKGKSYP
FEAHFVHADKDGNLAVIGVIFKEGKKNPIIEKIWENLPEAGKTIK
LAHKINAYDLLPKKKKYYRYSGSLTTPPCSEGVRWIVMEEEMELS
KEQIEKFRKLMGGDTNRPVQPLNARMIMEMD
(DNA)
SEQ ID NO: 12
ATGAAACAACAGAAAAGACTGTATGCACGCCTGCTTACATTACTG
TTTGCTCTTATTTTTCTTTTACCGCATTCAGCAGCGGCTGCCGG
CGGAGTTGGACATTGGTCTTATCATGGCGAAACAGGACCGCAACA
TTGGGGCGATCTGAAAAACGAATACATCATGTGCAAAATCGGCAA
AAACCAGTCACCGGTGGATATTAGCAGAATCGTCGAAGCTGAACT
TGAAAAAATCAAAATCAACTATTCAAGCGGCGGATCTTCAATCAC
AAACAACGGACATACAATCAAAGTTTCTTATGAACCGGGATCATA
TATTATCGTGGATGGCATTCGCTTTGAATTAAAACAATTTCATTT
TCATGCCCCGAGCGAACATACAATCAAAGGCAAATCTTATCCGTT
TGAAGCACATTTTGTCCATGCGGATAAAGATGGCAATCTGGCAGT
TATTGGAGTGATCTTTAAAGAAGGCAAGAAAAATCCGATCATCGA
AAAAATTTGGGAAAACTTACCGGAAGCGGGCAAAACAATCAAACT
GGCTCATAAAATCAACGCCTATGATCTGCTTCCGAAAAAGAAAAA
ATACTACAGATACAGCGGATCTCTTACAACACCGCCGTGTTCAGA
AGGCGTCCGCTGGATTGTTATGGAAGAAGAAATGGAACTTAGCAA
AGAACAAATCGAAAAATTTAGAAAACTGATGGGCGGAGATACAAA
TAGACCGGTTCAGCCGTTAAACGCTCGCATGATTATGGAAATGGA
TTAA

SEQ ID NO: 13 and SEQ ID NO: 14

SEQ name: SPamyL-LOGACA-CA4mut2

LENGTH: 255 for PRT; 768 for DNA

TYPE: PRT; DNA

ORGANISM: Signal peptide SPamyL from Bacillus licheniformis; LOGACA-CA4mut2 modified from deep sea thermal vent.

(PRT)
SEQ ID NO: 13
MKQQKRLYARLLTLLFALIFLLPHSAAAAGGVGHWSYHGETGPQH
WGDLKNEYIMCKIGKNQSPVDISRIVECELEKIKINYSSGGSSI
TNNGHTIKVSYEPGSYIIVDGIRFELKQFHFHAPSEHTIKGKSYP
FEAHFVHADKDGNLAVIGVIFKEGKKNPIIEKIWENLPEAGKTIK
LAHKINAYDLLPKKKKYYRYSGSLTTPPCSEGVRWIVMEEEMELS
KEQIEKFRKLMGGDTNRPVQPLNARMIMEMD
(DNA)
SEQ ID NO: 14
ATGAAACAACAGAAAAGACTGTATGCACGCCTGCTTACATTACTG
TTTGCTCTTATTTTTCTTTTACCGCATTCAGCAGCGGCTGCCGG
CGGAGTTGGACATTGGTCTTATCATGGCGAAACAGGACCGCAACA
TTGGGGCGATCTGAAAAACGAATACATCATGTGCAAAATCGGCAA
AAACCAGTCACCGGTGGATATTAGCAGAATCGTCGAATGTGAACT
TGAAAAAATCAAAATCAACTATTCAAGCGGCGGATCTTCAATCAC
AAACAACGGACATACAATCAAAGTTTCTTATGAACCGGGATCATA
TATTATCGTGGATGGCATTCGCTTTGAATTAAAACAATTTCATTT
TCATGCCCCGAGCGAACATACAATCAAAGGCAAATCTTATCCGTT
TGAAGCACATTTTGTCCATGCGGATAAAGATGGCAATCTGGCAGT
TATTGGAGTGATCTTTAAAGAAGGCAAGAAAAATCCGATCATCGA
AAAAATTTGGGAAAACTTACCGGAAGCGGGCAAAACAATCAAACT
GGCTCATAAAATCAACGCCTATGATCTGCTTCCGAAAAAGAAAAA
ATACTACAGATACAGCGGATCTCTTACAACACCGCCGTGTTCAGA
AGGCGTCCGCTGGATTGTTATGGAAGAAGAAATGGAACTTAGCAA
AGAACAAATCGAAAAATTTAGAAAACTGATGGGCGGAGATACAAA
TAGACCGGTTCAGCCGTTAAACGCTCGCATGATTATGGAAATGGA
TTAA

SEQ ID NO: 15 and SEQ ID NO: 16

SEQ name: SPamyL-LOGACA-CA4mut3

LENGTH: 255 for PRT; 768 for DNA

TYPE: PRT; DNA

ORGANISM: Signal peptide SPamyL from Bacillus licheniformis; LOGACA-CA4mut3 modified from deep sea thermal vent.

(PRT)
SEQ ID NO: 15
MKQQKRLYARLLTLLFALIFLLPHSAAAAGGVGHWSYHGETGPQH
WGDLKNEYIMCKIGKNQSPVDISRIVEAELEKIKINYSSGGSSI
TNNGHTIKVSYEPGSYIIVDGIRFELKQFHFHAPSEHTIKGKSYP
FEAHFVHADKDGNLAVIGVIFKEGKKNPIIEKIWENLPEAGKTIK
LAHKINAYDLLPKKKKYYRYCGSLTTPPCSEGVRWIVMEEEMELS
KEQIEKFRKLMGGDTNRPVQPLNCRMIMEMD
(DNA)
SEQ ID NO: 16
ATGAAACAACAGAAAAGACTGTATGCACGCCTGCTTACATTACTG
TTTGCTCTTATTTTTCTTTTACCGCATTCAGCAGCGGCTGCCGG
CGGAGTTGGACATTGGTCTTATCATGGCGAAACAGGACCGCAACA
TTGGGGCGATCTGAAAAACGAATACATCATGTGCAAAATCGGCAA
AAACCAGTCACCGGTGGATATTAGCAGAATCGTCGAAGCTGAACT
TGAAAAAATCAAAATCAACTATTCAAGCGGCGGATCTTCAATCAC
AAACAACGGACATACAATCAAAGTTTCTTATGAACCGGGATCATA
TATTATCGTGGATGGCATTCGCTTTGAATTAAAACAATTTCATTT
TCATGCCCCGAGCGAACATACAATCAAAGGCAAATCTTATCCGTT
TGAAGCACATTTTGTCCATGCGGATAAAGATGGCAATCTGGCAGT
TATTGGAGTGATCTTTAAAGAAGGCAAGAAAAATCCGATCATCGA
AAAAATTTGGGAAAACTTACCGGAAGCGGGCAAAACAATCAAACT
GGCTCATAAAATCAACGCCTATGATCTGCTTCCGAAAAAGAAAAA
ATACTACAGATACTGCGGATCTCTTACAACACCGCCGTGTTCAGA
AGGCGTCCGCTGGATTGTTATGGAAGAAGAAATGGAACTTAGCAA
AGAACAAATCGAAAAATTTAGAAAACTGATGGGCGGAGATACAAA
TAGACCGGTTCAGCCGTTAAACTGTCGCATGATTATGGAAATGGA
TTAA

SEQ ID NO: 17 and SEQ ID NO: 18

SEQ name: SPamyL-LOGACA-CA4mut23

LENGTH: 255 for PRT; 768 for DNA

TYPE: PRT; DNA

ORGANISM: Signal peptide SPamyL from Bacillus licheniformis; LOGACA-CA4mut23 modified from deep sea thermal vent.

(PRT)
SEQ ID NO: 17
MKQQKRLYARLLTLLFALIFLLPHSAAAAGGVGHWSYHGETGPQH
WGDLKNEYIMCKIGKNQSPVDISRIVECELEKIKINYSSGGSSI
TNNGHTIKVSYEPGSYIIVDGIRFELKQFHFHAPSEHTIKGKSYP
FEAHFVHADKDGNLAVIGVIFKEGKKNPIIEKIWENLPEAGKTIK
LAHKINAYDLLPKKKKYYRYCGSLTTPPCSEGVRWIVMEEEMELS
KEQIEKFRKLMGGDTNRPVQPLNCRMIMEMD
(DNA)
SEQ ID NO: 18
ATGAAACAACAGAAAAGACTGTATGCACGCCTGCTTACATTACTG
TTTGCTCTTATTTTTCTTTTACCGCATTCAGCAGCGGCTGCCGG
CGGAGTTGGACATTGGTCTTATCATGGCGAAACAGGACCGCAACA
TTGGGGCGATCTGAAAAACGAATACATCATGTGCAAAATCGGCAA
AAACCAGTCACCGGTGGATATTAGCAGAATCGTCGAATGTGAACT
TGAAAAAATCAAAATCAACTATTCAAGCGGCGGATCTTCAATCAC
AAACAACGGACATACAATCAAAGTTTCTTATGAACCGGGATCATA
TATTATCGTGGATGGCATTCGCTTTGAATTAAAACAATTTCATTT
TCATGCCCCGAGCGAACATACAATCAAAGGCAAATCTTATCCGTT
TGAAGCACATTTTGTCCATGCGGATAAAGATGGCAATCTGGCAGT
TATTGGAGTGATCTTTAAAGAAGGCAAGAAAAATCCGATCATCGA
AAAAATTTGGGAAAACTTACCGGAAGCGGGCAAAACAATCAAACT
GGCTCATAAAATCAACGCCTATGATCTGCTTCCGAAAAAGAAAAA
ATACTACAGATACTGCGGATCTCTTACAACACCGCCGTGTTCAGA
AGGCGTCCGCTGGATTGTTATGGAAGAAGAAATGGAACTTAGCAA
AGAACAAATCGAAAAATTTAGAAAACTGATGGGCGGAGATACAAA
TAGACCGGTTCAGCCGTTAAACTGTCGCATGATTATGGAAATGGA
TTAA

SEQ ID NO: 19 and SEQ ID NO: 20

SEQ name: SPamyL-SspCA-CA5

LENGTH: 255 for PRT; 768 for DNA

TYPE: PRT; DNA

ORGANISM: Signal peptide SPamyL from Bacillus licheniformis; SspCA-CA5 from Sulfurihydrogenibium sp. strain YO3AOP1.

(PRT)
SEQ ID NO: 19
MKQQKRLYARLLTLLFALIFLLPHSAAAAEHEWSYEGEKGPEHWA
QLKPEFFWCKLKNQSPINIDKKYKVKANLPKLNLYYKTAKESEV
VNNGHTIQINIKEDNTLNYLGEKYQLKQFHFHTPSEHTIEKKSYP
LEIHFVHKTEDGKILVVGVMAKLGKTNKELDKILNVAPAEEGEKI
LDKNLNLNNLIPKDKRYMTYSGSLTTPPCTEGVRWIVLKKPISIS
KQQLEKLKSVMVNPNNRPVQEINSRWIIEGF
(DNA)
SEQ ID NO: 20
ATGAAACAACAGAAAAGACTGTATGCACGCCTGCTTACATTACTG
TTTGCTCTTATTTTTCTTTTACCGCATTCAGCAGCGGCTGCCGA
ACATGAATGGTCTTATGAAGGCGAAAAAGGACCGGAACATTGGGC
ACAACTGAAACCGGAATTTTTCTGGTGCAAACTTAAAAACCAGTC
ACCGATCAACATCGATAAAAAATACAAAGTTAAAGCTAACCTGCC
GAAACTGAACCTTTACTACAAAACAGCCAAAGAATCAGAAGTTGT
GAATAACGGACATACAATCCAAATCAACATCAAAGAAGATAACAC
ACTTAACTACCTGGGCGAAAAATACCAACTGAAACAGTTTCATTT
TCATACACCGAGCGAACATACAATCGAGAAAAAATCATACCCGCT
TGAAATCCATTTTGTCCATAAAACAGAAGATGGCAAAATCCTTGT
CGTTGGAGTTATGGCTAAACTGGGCAAAACAAACAAAGAATTAGA
TAAAATTCTGAACGTGGCACCGGCGGAAGAAGGAGAAAAAATCTT
AGATAAAAACCTGAACCTGAACAACCTGATCCCGAAAGATAAAAG
ATACATGACATACTCAGGAAGCCTTACAACACCGCCGTGTACAGA
AGGCGTTCGCTGGATCGTGCTGAAAAAACCGATCTCTATTTCAAA
ACAACAGCTGGAAAAACTTAAATCAGTGATGGTCAATCCGAATAA
CAGACCGGTCCAGGAAATTAACAGCCGCTGGATTATCGAAGGCTT
TTAA

SEQ ID NO: 21 and SEQ ID NO: 22

SEQ name: SPamyL-SspCA-CA5mut2

LENGTH: 255 for PRT; 768 for DNA

TYPE: PRT; DNA

ORGANISM: Signal peptide SPamyL from Bacillus licheniformis; SspCA-CA5mut2 modified from Sulfurihydrogenibium sp. strain YO3AOP1.

(PRT)
SEQ ID NO: 21
MKQQKRLYARLLTLLFALIFLLPHSAAAAEHEWSYEGEKGPEHWA
QLKPEFFWCKLKNQSPINIDKKYKVKCNLPKLNLYYKTAKESEV
VNNGHTIQINIKEDNTLNYLGEKYQLKQFHFHTPSEHTIEKKSYP
LEIHFVHKTEDGKILVVGVMAKLGKTNKELDKILNVAPAEEGEKI
LDKNLNLNNLIPKDKRYMTYSGSLTTPPCTEGVRWIVLKKPISIS
KQQLEKLKSVMVNPNNRPVQEINSRWIIEGF
(DNA)
SEQ ID NO: 22
ATGAAACAACAGAAAAGACTGTATGCACGCCTGCTTACATTACTG
TTTGCTCTTATTTTTCTTTTACCGCATTCAGCAGCGGCTGCCGA
ACATGAATGGTCTTATGAAGGCGAAAAAGGACCGGAACATTGGGC
ACAACTGAAACCGGAATTTTTCTGGTGCAAACTTAAAAACCAGTC
ACCGATCAACATCGATAAAAAATACAAAGTTAAATGTAACCTGCC
GAAACTGAACCTTTACTACAAAACAGCCAAAGAATCAGAAGTTGT
GAATAACGGACATACAATCCAAATCAACATCAAAGAAGATAACAC
ACTTAACTACCTGGGCGAAAAATACCAACTGAAACAGTTTCATTT
TCATACACCGAGCGAACATACAATCGAGAAAAAATCATACCCGCT
TGAAATCCATTTTGTCCATAAAACAGAAGATGGCAAAATCCTTGT
CGTTGGAGTTATGGCTAAACTGGGCAAAACAAACAAAGAATTAGA
TAAAATTCTGAACGTGGCACCGGCGGAAGAAGGAGAAAAAATCTT
AGATAAAAACCTGAACCTGAACAACCTGATCCCGAAAGATAAAAG
ATACATGACATACTCAGGAAGCCTTACAACACCGCCGTGTACAGA
AGGCGTTCGCTGGATCGTGCTGAAAAAACCGATCTCTATTTCAAA
ACAACAGCTGGAAAAACTTAAATCAGTGATGGTCAATCCGAATAA
CAGACCGGTCCAGGAAATTAACAGCCGCTGGATTATCGAAGGCTT
TTAA

SEQ ID NO: 23 and SEQ ID NO: 24

SEQ name: SPamyL-SspCA-CA5mut3

LENGTH: 255 for PRT; 768 for DNA

TYPE: PRT; DNA

ORGANISM: Signal peptide SPamyL from Bacillus licheniformis; SspCA-CA5mut3 modified from Sulfurihydrogenibium sp. strain YO3AOP1.

(PRT)
SEQ ID NO: 23
MKQQKRLYARLLTLLFALIFLLPHSAAAAEHEWSYEGEKGPEHWA
QLKPEFFWCKLKNQSPINIDKKYKVKANLPKLNLYYKTAKESEV
VNNGHTIQINIKEDNTLNYLGEKYQLKQFHFHTPSEHTIEKKSYP
LEIHFVHKTEDGKILVVGVMAKLGKTNKELDKILNVAPAEEGEKI
LDKNLNLNNLIPKDKRYMTYCGSLTTPPCTEGVRWIVLKKPISIS
KQQLEKLKSVMVNPNNRPVQEINCRWIIEGF
(DNA)
SEQ ID NO: 24
ATGAAACAACAGAAAAGACTGTATGCACGCCTGCTTACATTACTG
TTTGCTCTTATTTTTCTTTTACCGCATTCAGCAGCGGCTGCCGA
ACATGAATGGTCTTATGAAGGCGAAAAAGGACCGGAACATTGGGC
ACAACTGAAACCGGAATTTTTCTGGTGCAAACTTAAAAACCAGTC
ACCGATCAACATCGATAAAAAATACAAAGTTAAAGCTAACCTGCC
GAAACTGAACCTTTACTACAAAACAGCCAAAGAATCAGAAGTTGT
GAATAACGGACATACAATCCAAATCAACATCAAAGAAGATAACAC
ACTTAACTACCTGGGCGAAAAATACCAACTGAAACAGTTTCATTT
TCATACACCGAGCGAACATACAATCGAGAAAAAATCATACCCGCT
TGAAATCCATTTTGTCCATAAAACAGAAGATGGCAAAATCCTTGT
CGTTGGAGTTATGGCTAAACTGGGCAAAACAAACAAAGAATTAGA
TAAAATTCTGAACGTGGCACCGGCGGAAGAAGGAGAAAAAATCTT
AGATAAAAACCTGAACCTGAACAACCTGATCCCGAAAGATAAAAG
ATACATGACATACTGCGGAAGCCTTACAACACCGCCGTGTACAGA
AGGCGTTCGCTGGATCGTGCTGAAAAAACCGATCTCTATTTCAAA
ACAACAGCTGGAAAAACTTAAATCAGTGATGGTCAATCCGAATAA
CAGACCGGTCCAGGAAATTAACTGTCGCTGGATTATCGAAGGCTT
TTAA

SEQ ID NO: 25 and SEQ ID NO: 26

SEQ name: SPamyL-SspCA-CA5mut23

LENGTH: 255 for PRT; 768 for DNA

TYPE: PRT; DNA

ORGANISM: Signal peptide SPamyL from Bacillus licheniformis; SspCA-CA5mut23 modified from Sulfurihydrogenibium sp. strain YO3AOP1.

(PRT)
SEQ ID NO: 25
MKQQKRLYARLLTLLFALIFLLPHSAAAAEHEWSYEGEKGPEHWA
QLKPEFFWCKLKNQSPINIDKKYKVKCNLPKLNLYYKTAKESEV
VNNGHTIQINIKEDNTLNYLGEKYQLKQFHFHTPSEHTIEKKSYP
LEIHFVHKTEDGKILVVGVMAKLGKTNKELDKILNVAPAEEGEKI
LDKNLNLNNLIPKDKRYMTYCGSLTTPPCTEGVRWIVLKKPISIS
KQQLEKLKSVMVNPNNRPVQEINCRWIIEGF
(DNA)
SEQ ID NO: 26
ATGAAACAACAGAAAAGACTGTATGCACGCCTGCTTACATTACTG
TTTGCTCTTATTTTTCTTTTACCGCATTCAGCAGCGGCTGCCGA
ACATGAATGGTCTTATGAAGGCGAAAAAGGACCGGAACATTGGGC
ACAACTGAAACCGGAATTTTTCTGGTGCAAACTTAAAAACCAGTC
ACCGATCAACATCGATAAAAAATACAAAGTTAAATGTAACCTGCC
GAAACTGAACCTTTACTACAAAACAGCCAAAGAATCAGAAGTTGT
GAATAACGGACATACAATCCAAATCAACATCAAAGAAGATAACAC
ACTTAACTACCTGGGCGAAAAATACCAACTGAAACAGTTTCATTT
TCATACACCGAGCGAACATACAATCGAGAAAAAATCATACCCGCT
TGAAATCCATTTTGTCCATAAAACAGAAGATGGCAAAATCCTTGT
CGTTGGAGTTATGGCTAAACTGGGCAAAACAAACAAAGAATTAGA
TAAAATTCTGAACGTGGCACCGGCGGAAGAAGGAGAAAAAATCTT
AGATAAAAACCTGAACCTGAACAACCTGATCCCGAAAGATAAAAG
ATACATGACATACTGCGGAAGCCTTACAACACCGCCGTGTACAGA
AGGCGTTCGCTGGATCGTGCTGAAAAAACCGATCTCTATTTCAAA
ACAACAGCTGGAAAAACTTAAATCAGTGATGGTCAATCCGAATAA
CAGACCGGTCCAGGAAATTAACTGTCGCTGGATTATCGAAGGCTT
TTAA

SEQ ID NO: 27 and SEQ ID NO: 28

SEQ name: SPamyL-SazCA-CA6FL

LENGTH: 266 for PRT; 801 for DNA

TYPE: PRT; DNA

ORGANISM: Signal peptide SPamyL from Bacillus licheniformis; SazCA-CA6FL from Sulfurihydrogenibium azorense

(PRT)
SEQ ID NO: 27
MKQQKRLYARLLTLLFALIFLLPHSAAAAGEHAILQKNAEVHHWS
YEGENGPENWAKLNPEYFWCNLKNQSPVDISDNYKVHAKLEKLH
INYNKAVNPEIVNNGHTIQVNVLEDFKLNIKGKEYHLKQFHFHAP
SEHTVNGKYYPLEMHLVHKDKDGNIAVIGVFFKEGKANPELDKVF
KNALKEEGSKVFDGSININALLPPVKNYYTYSGSLTTPPCTEGVL
WIVLKQPITASKQQIELFKSIMKHNNNRPTQPINSRYILESN
(DNA)
In an embodiment, also referred to as:
>2_Gene6-pH43-SPamyL-fullSazCA
SEQ ID NO: 28
ATGAAACAACAGAAAAGACTGTATGCACGCCTGCTTACATTACTG
TTTGCTCTTATTTTTCTTTTACCGCATTCAGCAGCGGCTGCCGG
CGAACATGCAATTCTGCAGAAAAATGCGGAAGTCCATCATTGGAG
CTATGAAGGCGAAAACGGACCGGAAAATTGGGCCAAACTGAACCC
GGAATACTTTTGGTGCAACCTTAAAAACCAGTCTCCGGTCGATAT
TTCAGATAACTACAAAGTTCATGCCAAACTGGAAAAACTGCATAT
CAACTACAACAAAGCAGTTAACCCGGAAATTGTGAATAACGGACA
TACAATCCAAGTTAACGTGTTAGAAGATTTTAAACTGAACATCAA
AGGCAAAGAATACCATCTTAAACAGTTTCATTTTCATGCTCCGTC
TGAACATACAGTGAACGGCAAATATTATCCGCTTGAAATGCATCT
GGTCCATAAAGATAAAGATGGCAACATTGCAGTCATCGGAGTTTT
CTTTAAAGAAGGCAAAGCGAACCCGGAACTTGATAAAGTTTTTAA
AAACGCTCTGAAAGAAGAAGGAAGCAAAGTGTTTGATGGCTCTAT
TAACATCAATGCGCTGCTTCCGCCGGTTAAAAACTACTACACATA
CTCAGGAAGCTTAACAACACCGCCGTGTACAGAAGGCGTGCTGTG
GATTGTCCTTAAACAACCGATCACAGCTTCTAAACAACAGATTGA
ACTGTTTAAATCAATCATGAAACATAACAACAATAGACCGACACA
GCCGATTAACTCACGCTATATCCTGGAAAGCAATTAA

SEQ ID NO: 29 and SEQ ID NO: 30

SEQ name: SPamyL-SazCA-CA6FLmut2

LENGTH: 266 for PRT; 801 for DNA

TYPE: PRT; DNA

ORGANISM: Signal peptide SPamyL from Bacillus licheniformis; SazCA-CA6FLmut2 modified from Sulfurihydrogenibium azorense

(PRT)
SEQ ID NO: 29
MKQQKRLYARLLTLLFALIFLLPHSAAAAGEHAILQKNAEVHHWS
YEGENGPENWAKLNPEYFWCNLKNQSPVDISDNYKVHCKLEKLH
INYNKAVNPEIVNNGHTIQVNVLEDFKLNIKGKEYHLKQFHFHAP
SEHTVNGKYYPLEMHLVHKDKDGNIAVIGVFFKEGKANPELDKVF
KNALKEEGSKVFDGSININALLPPVKNYYTYSGSLTTPPCTEGVL
WIVLKQPITASKQQIELFKSIMKHNNNRPTQPINSRYILESN
(DNA)
SEQ ID NO: 30
ATGAAACAACAGAAAAGACTGTATGCACGCCTGCTTACATTACTG
TTTGCTCTTATTTTTCTTTTACCGCATTCAGCAGCGGCTGCCGG
CGAACATGCAATTCTGCAGAAAAATGCGGAAGTCCATCATTGGAG
CTATGAAGGCGAAAACGGACCGGAAAATTGGGCCAAACTGAACCC
GGAATACTTTTGGTGCAACCTTAAAAACCAGTCTCCGGTCGATAT
TTCAGATAACTACAAAGTTCATTGTAAACTGGAAAAACTGCATAT
CAACTACAACAAAGCAGTTAACCCGGAAATTGTGAATAACGGACA
TACAATCCAAGTTAACGTGTTAGAAGATTTTAAACTGAACATCAA
AGGCAAAGAATACCATCTTAAACAGTTTCATTTTCATGCTCCGTC
TGAACATACAGTGAACGGCAAATATTATCCGCTTGAAATGCATCT
GGTCCATAAAGATAAAGATGGCAACATTGCAGTCATCGGAGTTTT
CTTTAAAGAAGGCAAAGCGAACCCGGAACTTGATAAAGTTTTTAA
AAACGCTCTGAAAGAAGAAGGAAGCAAAGTGTTTGATGGCTCTAT
TAACATCAATGCGCTGCTTCCGCCGGTTAAAAACTACTACACATA
CTCAGGAAGCTTAACAACACCGCCGTGTACAGAAGGCGTGCTGTG
GATTGTCCTTAAACAACCGATCACAGCTTCTAAACAACAGATTGA
ACTGTTTAAATCAATCATGAAACATAACAACAATAGACCGACACA
GCCGATTAACTCACGCTATATCCTGGAAAGCAATTAA

SEQ ID NO: 31 and SEQ ID NO: 32

SEQ name: SPamyL-SazCA-CA6FLmut3

LENGTH: 266 for PRT; 801 for DNA

TYPE: PRT; DNA

ORGANISM: Signal peptide SPamyL from Bacillus licheniformis; SazCA-CA6FLmut3 modified from Sulfurihydrogenibium azorense

(PRT)
SEQ ID NO: 31
MKQQKRLYARLLTLLFALIFLLPHSAAAAGEHAILQKNAEVHHWS
YEGENGPENWAKLNPEYFWCNLKNQSPVDISDNYKVHAKLEKLH
INYNKAVNPEIVNNGHTIQVNVLEDFKLNIKGKEYHLKQFHFHAP
SEHTVNGKYYPLEMHLVHKDKDGNIAVIGVFFKEGKANPELDKVF
KNALKEEGSKVFDGSININALLPPVKNYYTYCGSLTTPPCTEGVL
WIVLKQPITASKQQIELFKSIMKHNNNRPTQPINCRYILESN
(DNA)
SEQ ID NO: 32
ATGAAACAACAGAAAAGACTGTATGCACGCCTGCTTACATTACTG
TTTGCTCTTATTTTTCTTTTACCGCATTCAGCAGCGGCTGCCGG
CGAACATGCAATTCTGCAGAAAAATGCGGAAGTCCATCATTGGAG
CTATGAAGGCGAAAACGGACCGGAAAATTGGGCCAAACTGAACCC
GGAATACTTTTGGTGCAACCTTAAAAACCAGTCTCCGGTCGATAT
TTCAGATAACTACAAAGTTCATGCCAAACTGGAAAAACTGCATAT
CAACTACAACAAAGCAGTTAACCCGGAAATTGTGAATAACGGACA
TACAATCCAAGTTAACGTGTTAGAAGATTTTAAACTGAACATCAA
AGGCAAAGAATACCATCTTAAACAGTTTCATTTTCATGCTCCGTC
TGAACATACAGTGAACGGCAAATATTATCCGCTTGAAATGCATCT
GGTCCATAAAGATAAAGATGGCAACATTGCAGTCATCGGAGTTTT
CTTTAAAGAAGGCAAAGCGAACCCGGAACTTGATAAAGTTTTTAA
AAACGCTCTGAAAGAAGAAGGAAGCAAAGTGTTTGATGGCTCTAT
TAACATCAATGCGCTGCTTCCGCCGGTTAAAAACTACTACACATA
CTGCGGAAGCTTAACAACACCGCCGTGTACAGAAGGCGTGCTGTG
GATTGTCCTTAAACAACCGATCACAGCTTCTAAACAACAGATTGA
ACTGTTTAAATCAATCATGAAACATAACAACAATAGACCGACACA
GCCGATTAACTGTCGCTATATCCTGGAAAGCAATTAA

SEQ ID NO: 33 and SEQ ID NO: 34

SEQ name: SPamyL-SazCA-CA6FLmut23

LENGTH: 266 for PRT; 801 for DNA

TYPE: PRT; DNA

ORGANISM: Signal peptide SPamyL from Bacillus licheniformis; SazCA-CA6FLmut23 modified from Sulfurihydrogenibium azorense

(PRT)
SEQ ID NO: 33
MKQQKRLYARLLTLLFALIFLLPHSAAAAGEHAILQKNAEVHHWS
YEGENGPENWAKLNPEYFWCNLKNQSPVDISDNYKVHCKLEKLH
INYNKAVNPEIVNNGHTIQVNVLEDFKLNIKGKEYHLKQFHFHAP
SEHTVNGKYYPLEMHLVHKDKDGNIAVIGVFFKEGKANPELDKVF
KNALKEEGSKVEDGSININALLPPVKNYYTYCGSLTTPPCTEGVL
WIVLKQPITASKQQIELFKSIMKHNNNRPTQPINCRYILESN
(DNA)
SEQ ID NO: 34
ATGAAACAACAGAAAAGACTGTATGCACGCCTGCTTACATTACTG
TTTGCTCTTATTTTTCTTTTACCGCATTCAGCAGCGGCTGCCGG
CGAACATGCAATTCTGCAGAAAAATGCGGAAGTCCATCATTGGAG
CTATGAAGGCGAAAACGGACCGGAAAATTGGGCCAAACTGAACCC
GGAATACTTTTGGTGCAACCTTAAAAACCAGTCTCCGGTCGATAT
TTCAGATAACTACAAAGTTCATTGTAAACTGGAAAAACTGCATAT
CAACTACAACAAAGCAGTTAACCCGGAAATTGTGAATAACGGACA
TACAATCCAAGTTAACGTGTTAGAAGATTTTAAACTGAACATCAA
AGGCAAAGAATACCATCTTAAACAGTTTCATTTTCATGCTCCGTC
TGAACATACAGTGAACGGCAAATATTATCCGCTTGAAATGCATCT
GGTCCATAAAGATAAAGATGGCAACATTGCAGTCATCGGAGTTTT
CTTTAAAGAAGGCAAAGCGAACCCGGAACTTGATAAAGTTTTTAA
AAACGCTCTGAAAGAAGAAGGAAGCAAAGTGTTTGATGGCTCTAT
TAACATCAATGCGCTGCTTCCGCCGGTTAAAAACTACTACACATA
CTGCGGAAGCTTAACAACACCGCCGTGTACAGAAGGCGTGCTGTG
GATTGTCCTTAAACAACCGATCACAGCTTCTAAACAACAGATTGA
ACTGTTTAAATCAATCATGAAACATAACAACAATAGACCGACACA
GCCGATTAACTGTCGCTATATCCTGGAAAGCAATTAA

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

What is claimed is:

1. A non-naturally occurring carbonic anhydrase comprising at least one mutation that results in the substitution of at least one cysteine for at least one amino acid in a naturally occurring carbonic anhydrase; and wherein the non-naturally occurring carbonic anhydrase has increased activity at a temperature of greater than about 60 degrees Celsius when compared to the naturally occurring carbonic anhydrase.

2. The non-naturally occurring carbonic anhydrase of claim 1 wherein the increased activity is for more than about 30 minutes, 1 hour, 2 hours, 3 hours, 4 hours, 5 hours, 6 hours, 24 hours, 44 hours, 48 hours, and 92 hours.

3. The non-naturally occurring carbonic anhydrase of claim 1 wherein the increased activity is at a temperature greater than 65, 70, 75, 80, 85 or 90 degrees Celsius.

4. The non-naturally occurring carbonic anhydrase of claim 1 wherein a nucleotide sequence encoding the non-naturally occurring carbonic anhydrase comprises a sequence that is greater than 70% identical to a sequence selected from the group consisting of SEQ ID NO: 6, SEQ ID NO: 8, SEQ ID NO: 22, SEQ ID NO: 24 and SEQ ID NO: 26.

5. The non-naturally occurring carbonic anhydrase of claim 1 comprising an amino acid sequence that is greater than 70% identical to a sequence selected from the group consisting of SEQ ID NO: 5, SEQ ID NO: 7, SEQ ID NO: 21, SEQ ID NO: 23 and SEQ ID NO: 25.

6. A method for CO₂ separation and CO₂ capture comprising the step of reacting CO₂ with a non-naturally occurring carbonic anhydrase comprising at least one mutation that results in the substitution of at least one cysteine for at least one amino acid in a naturally occurring carbonic anhydrase; and wherein the non-naturally occurring carbonic anhydrase has increased activity at a temperature of greater than about 60 degrees Celsius when compared to the naturally occurring carbonic anhydrase.

7. The method of claim 6 wherein the step of reacting CO₂ with non-naturally occurring carbonic anhydrase is for more than about 30 minutes, 1 hour, 2 hours, 3 hours, 4 hours, 5 hours, 6 hours, 24 hours, 44 hours, 48 hours, and 92 hours.

8. The method of claim 6 wherein the step of reacting CO₂ with the non-naturally occurring carbonic anhydrase is at a temperature greater than 65, 70, 75, 80, 85 or 90 degrees Celsius.

9. The method of claim 6 wherein the non-naturally occurring carbonic anhydrase comprises a nucleotide sequence that is greater than 70% identical to a sequence selected from the group consisting of SEQ ID NO: 6, SEQ ID NO: 8, SEQ ID NO: 22, SEQ ID NO: 24 and SEQ ID NO: 26.

10. The method of claim 6 wherein the non-naturally occurring carbonic anhydrase comprises an amino acid sequence that is greater than 70% identical to a sequence selected from the group consisting of SEQ ID NO: 5, SEQ ID NO: 7, SEQ ID NO: 21, SEQ ID NO: 23 and SEQ ID NO: 25.

11. A system for CO₂ separation and CO₂ capture comprising s non-naturally occurring carbonic anhydrase comprising at least one mutation that results in the substitution of at least one cysteine for at least one amino acid in a naturally occurring carbonic anhydrase; and wherein the non-naturally occurring carbonic anhydrase has increased activity at a temperature of greater than about 60 degrees Celsius when compared to the naturally occurring carbonic anhydrase; and wherein the system further comprises a support wherein the with the non-naturally occurring carbonic anhydrase is immobilized to the support; and wherein the non-naturally occurring carbonic anhydrase is contacted with CO₂.

12. The system of claim 11 wherein the non-naturally occurring carbonic anhydrase has increased activity for more than about 30 minutes, 1 hour, 2 hours, 3 hours, 4 hours, 5 hours, 6 hours, 24 hours, 44 hours, 48 hours, and 92 hours.

13. The system of claim 11 wherein the non-naturally occurring carbonic anhydrase reacts with CO₂ at a temperature greater than 65, 70, 75, 80, 85 or 90 degrees Celsius.

14. The system of claim 11 wherein the CO₂ to be separated and captured results from the combustion of fossil fuels or biomass.

15. The system of claim 11 further comprising a carbon capture unit wherein the carbon capture unit comprises an immobilized biocatalyst comprising an amino acid sequence that is greater than 70% identical to a sequence selected from the group consisting of SEQ ID NO: 5, SEQ ID NO: 7, SEQ ID NO: 21, SEQ ID NO: 23 and SEQ ID NO: 25.
